SEQUENCE LISTING (10 sequences)

SEQ ID NO: 1 - DNA, 20 bp, Artificial Sequence
  A synthetic sequence
taatgtgtgggcttgggatt

SEQ ID NO: 2 - DNA, 24 bp, Artificial Sequence
  A synthetic sequence
tgcatatggaaaatacagaggcta

SEQ ID NO: 3 - DNA, 26 bp, Artificial Sequence
  A synthetic sequence
attcgattccattcgatgatgattcc

SEQ ID NO: 4 - DNA, 26 bp, Artificial Sequence
  A synthetic sequence
ggaaccgaatgaatcctcattgaatg

SEQ ID NO: 5 - DNA, 27 bp, Artificial Sequence
  A synthetic sequence
atcagagaatactacaaacacctctac

SEQ ID NO: 6 - DNA, 21 bp, Artificial Sequence
  A synthetic sequence
agagtgtatgtgtcgaggaat

SEQ ID NO: 7 - DNA, 22 bp, Artificial Sequence
  A synthetic sequence
acaaaagcactgaaaagcctgc

SEQ ID NO: 8 - DNA, 23 bp, Artificial Sequence
  A synthetic sequence
tccagtctactccgtaatctcgt

SEQ ID NO: 9 - DNA, 31 bp, Artificial Sequence
  A synthetic sequence
tgtgaaagactttcaggaagatgtagaaaaa

SEQ ID NO: 10 - DNA, 25 bp, Artificial Sequence
  A synthetic sequence
gagggagtggcatctttcttaaatg

(19) United States
(12) Patent Application Publication
(10) Pub. No.: US 2023/0383358 A1
(43) Pub. Date: Nov. 30, 2023

(54) REPETITIVE ELEMENT DNAS AND USES THEREOF

(71) Applicant: Children's Hospital Los Angeles, Los Angeles, CA (US)

(72) Inventors: David Cobrinik, Los Angeles, CA (US); Linda Cambier, Los Angeles, CA (US)

(21) Appl. No.: 18/031,534

(22) PCT Filed: Oct. 16, 2021
(86) PCT No.: PCT/US2021/071910

§ 371 Date: Apr. 12, 2023

Related U.S. Application Data
(60) Provisional application No. 63/093,004, filed on Oct. 16, 2020.

(51) Int. Cl. C12Q 1/6886; C12Q 1/6806

(52) U.S. Cl. CPC C12Q 1/6886; C12Q 1/6806; G01N 2800/7028

(57) ABSTRACT

Provided herein is a blood test for the detection of a marker of osteosarcoma and other cancers.

### PRIORITY CLAIM

This application claims the benefit of priority of U.S. Provisional Patent Application No. 63/093,004, filed on Oct. 16, 2020, the benefit of priority of which is claimed hereby, and which is incorporated by reference herein in its entirety.

### BACKGROUND OF THE INVENTION

Osteosarcoma (OS) is the most common malignant bone tumor of children, adolescents,

and young adults, representing approximately 1% of newly diagnosed cancers in adults, and 3-5% in children (1,2). With current treatment regimens, patients with non-metastatic OS have five-year survival rates above 65% whereas the ˜25% of patients presenting with metastases have a five-year survival of less than 20% (3,4). As such, early detection of OS prior to metastasis could significantly improve outcomes.

Early detection is especially needed in individuals who are predisposed to OS either genetically or through iatrogenic exposures. OS occurs at increased rates in several monogenic hereditary cancer syndromes such as retinoblastoma (RBI) (5), Li-Fraumeni syndrome (TP53), Bloom syndrome (RECQL2), Werner syndrome (RECQL3), and Rothmund-Thomson syndrome (RECQL4). OS also occurs with increased frequency in children exposed to radiation or alkylating agents, in Diamond-Blackfan anaemia patients and in adults with bone disorders such as Paget's Disease. Combined genetic predisposition and exposure to DNA damaging agents confers particularly high risk; for example, relative risk for children with hereditary retinoblastoma increased from ˜69 without such treatments to ˜302 for radiotherapy and ˜539 for radiotherapy plus chemotherapy in the largest treatment-stratified analysis (5).

The need for OS biomarkers is reflected in a large yet inconclusive literature. Early

studies focusing on bone markers such as alkaline phosphatase showed highly variable increases in OS patients (6). Later proteomic studies revealed two as-yet uncharacterized OS-associated proteins (7) whereas studies of miRNAs showed variable results (8-10). A recent study identified 56 miRs that were upregulated in pre-treatment OS patient plasma (11); however, among the top candidates (miR-21, miR-221, and miR-106a), levels increased by only ˜2.4-8-fold and sensitivity was at best ˜85%. An alternative approach is to detect aneuploidy via cell-free DNA (cfDNA) whole genome sequencing, yet at present this has limited sensitivity (12) due to the dilution of tumor with non-tumor cfDNA. Currently no biomarkers have been shown to reliably detect naïve pre-symptomatic OS in predisposed individuals (13).

Beyond OS, many other cancers lack biomarkers with sufficient sensitivity, specificity, and low cost to enable medically beneficial and feasible cancer screening in cancer-predisposed patients and the general public. For example, a recently developed cancer screening approach (CancerSEEK) involves targeted sequencing and detection of single nucleotide variants (SNVs) and small insertions or deletions (INDELS) in ˜500 cancer-related genes (e.g., oncogenes and tumor suppressor genes) in circulating tumor DNA (ctDNA). However, this approach cannot detect cancers that lack SNVs or INDELS in the pre-selected gene panel, such as the ˜50% of OS with causative chromosome structure changes and may be prohibitively expensive to deploy on a population-wide basis. Similarly, circulating tumor cells (CTCs) can be a good indicator of cancer, but CTC screens may fail to detect small incipient tumors and can require complex and expensive technology. miRNA biomarkers have also been explored yet have low or inconsistent sensitivity in different studies.

Beyond early cancer detection, there is a need for more sensitive monitoring of cancer treatment responses and relapse. In many cancers, response and relapse are monitored by imaging, which might not detect the smallest, most treatable lesions, or by reappearance of symptoms, which might occur only after a tumor has advanced. As for cancer screening, cancer treatment response and relapse may be monitored by ctDNA sequencing, CTC detection, and circulating miRNAs, yet the same sensitivity and cost drawbacks as noted for screening may apply. Thus, there is a need to develop a simpler, faster, less expensive, and more sensitive approaches for early cancer detection and therapy response and relapse monitoring. Optimally, a new approach will detect a range of cancers, so it may be widely deployed, and positive results may be followed by appropriate targeted diagnostics and therapeutic interventions.

### SUMMARY OF THE INVENTION

Provided herein is a novel type of blood test for the detection of a novel marker of osteosarcoma and other cancers. Specifically, the test enables the detection of an increased level of a specific category of human genomic DNA, the repetitive element (RE) DNAs, in serum or plasma of people with cancers (including serum extracellular vesicle-associated repetitive element DNAs as candidate osteosarcoma biomarkers). The increase may be assessed either in terms of the total RE DNA levels or in terms of the RE DNA relative to other sequences in the same nucleic acid preparations.

The detection of the increased RE DNAs in serum or plasma requires that the RE DNAs are isolated by specific methods that have not previously been used for this or related purposes, and which exploit the novel finding that RE DNAs co-purify with extracellular vesicles (EVs) using specific biochemical methods. The novel method involves a) specific methods to isolate/enrich EVs and associated material and/or a method to isolate RE DNA; b) a specific method to isolate ‘small nucleic acids’ from the EV preparations; and c) quantitation of i) RE DNA sequences or ii) the ratio of RE DNA to other nucleic acid sequences. When quantitating the ratio of RE DNA to other nucleic acid sequences, the method may involve concurrent analysis of EV-associated RE DNA and non-RE RNA sequences.

One aspect is the combined use of the EV isolation step and the small nucleic acid isolation step. Omitting either the EV isolation step or using a standard DNA isolation method after EV isolation would not selectively enrich for tumor associated RE DNAs (or enrich to a lesser extent) or enable detection of higher RE DNA levels (total or relative to non-RE sequences) in individuals with cancer. The combined use of an EV isolation step plus a small nucleic acid isolation step has not previously been used to detect differentially represented DNA sequences in fluids from cancer-bearing versus normal individuals. The utility of the test employing this aspect was demonstrated by the finding that the test can discriminate serum samples from individuals with vs without osteosarcoma (FIG. 3).

Another aspect is the quantitation of the proportion of RE DNA relative to the total sequences in the EV-associated nucleic acid preparation, which can improve cancer detection sensitivity. The total sequences in the nucleic acid preparation include RE and non-RE genomic DNA (gDNA) sequences as well as RNA sequences. The “RE DNA proportion test” examines the proportion of RE DNA sequences among total sequences, which may be determined by a) reverse transcription of RNA into cDNA, b) co-amplification of EV-associated gDNA and the reverse-transcribed cDNA, and c) detection of RE DNA and non-RE DNA. As examples, the proportion of RE DNA a) may be defined by massively parallel sequencing and expressed as read counts-per-million (CPM), or b) may be defined by capture of the amplified total sequences by a limiting quantity of immobilized capture probes complementary to the DNA amplification primers, followed by probing the captured sequences with fluorescent oligodeoxynucleotides complementary to RE or non-RE sequences of interest. The latter approach measures the proportion of the total sequences comprised of each RE or non-RE sequence, rather than absolute levels, because the capture probes are present in limiting quantity relative to the amplification products and thus capture representative proportions of sequences of interest (as illustrated in FIG. 9). The method can be used to measure the proportion of total reads comprising specific RE DNA sequences or multiple different RE DNA sequences. Other methods of measuring the proportion of RE DNA sequences among total sequences (besides the examples in (a) and (b)) are possible to those skilled in the art but are covered because the concept of this proportion test is novel. The utility of measuring the proportion of RE DNA sequences relative to total sequences is illustrated by the high ROC curve AUC values comparing sera from OS patients versus healthy controls, with HSATII AUC=1.0; LINE1 family AUC=0.986; and base means of the 15 most significantly over-represented REs AUC=0.99 (FIGS. 7a-c). There are no prior descriptions of a) using the proportion of RE DNA relative to total (both RNA and DNA) sequences as a cancer diagnostic, or b) concurrent amplification and quantitation of RNA and RE DNA sequences in the same sample in a clinical test.

An additional aspect is the quantitation of the ratio of one or more RE DNA sequences to certain down-regulated (or under-represented) non-RE sequence, which can further improve cancer detection. The non-RE sequences may consist of RNAs that co-purify with EVs and whose proportion of total reads declines as RE DNAs increase in cancer patient serum or plasma (FIG. 7 panel c, top, right). The utility of measuring the ratio of RE DNA sequences to down-regulated non-RE sequence is illustrated in a ROC curve comparing base means of 15 over-represented RE to 138 under-represented non-RE sequences, with AUC=1.0, whereas measuring the 15 RE DNAs as a proportion of total reads had AUC=0.99 (FIGS. 7c, d). This improved discrimination of OS vs, control sera may decrease false positives in a screening test. This ratio test may be used to evaluate ratios of multiple over- and under-represented RE DNAs (as in FIG. 7 panel d) or ratios of single specific over-represented and/or under-represented sequences (not shown) using either next generation sequencing or RT−PCR+qPCR analyses. There is no previous description of a test that compares the ratio of RE DNA to specific non-RE sequences.

Thus, the test may be formatted in several ways as described above (PCR to directly quantitate RE DNA levels; sequencing or hybridization to define the RE DNA proportion; or comparing the RE DNA and non-RE sequence ratio) to improve utility as may be appropriate to different applications. The test may also be used in several ways, including a) to detect a new osteosarcoma (or other cancers) before the cancer would be detected through the usual clinical presentation. Thus, the method could be used such as in a cancer-screening regimen in individuals who are predisposed to osteosarcoma (or other cancers); b) to monitor therapy response, which is reflected in altered levels of the EV-associated RE-DNAs and can be a prognostic indicator; and/or c) to monitor tumor recurrence prior to its clinical appearance, in patients who are effectively treated but at risk for relapse. Of note, the test can be used in combination with other markers of OS or other cancers. As an example of such a marker, named herein as ‘p90,’ which was elevated in serum density gradient EV fractions in each of 7 OS patients versus 7 controls (FIG. 8).

A variation of the method in which the tumor RE DNA is enriched based on tumor specific epigenetic features can also be carried out to provide further enrichment and/or sensitivity.

### BRIEF DESCRIPTION OF THE DRAWINGS

FIGS. 1A-1E. Patient EV preparation characteristics. (a, b) Representative control (a) and OS (b) particle size distributions in preparations used for nucleic acid extraction and sequencing, as defined by nanoparticle tracking (NanoSight). (c, d) Dot plots representing EV concentration of preparations from sera of OS patients (n=12) and controls from hereditary retinoblastoma sibling (HRC, n=4), and unrelated healthy (HC, n=8) and color-coded by source (c) and sex (d). Lines represent mean and standard deviation. Groups were compared using two-tailed, unpaired, Mann Whitney U test; *P<0.05. (e) Dot plots of EV concentration versus donor age and color coded according to OS type. Spearman's correlation (r) between EV concentration and donor age was not significant (p=0.32).

FIGS. 2A-2D. Over-representation of repetitive elements in OS EV-associated sequences. MA plot for sequence features differentially represented in control and OS serum EV preparations as defined by Tetranscripts analysis 1. (a) Differentially represented single-copy genes and repetitive elements (REs), significantly differentially represented in red. (b) Differentially represented single-copy genes, significantly differentially represented in red. (c) Differentially represented single-copy genes in blue and REs in red. (d) Differentially represented REs, significantly differentially represented in red. Arrows, the significantly over-represented HSATI and Charlie 3. Arrowheads, significantly under-represented REs. Significantly differentially represented defined by FDR <0.05, Wald test.

FIGS. 3A-3C. Over-representation of repetitive elements in OS compared to control EV preparations in a validation cohort. (a) Violin plots representing relative abundance of HSATI, HSATII, L1P1 and Charlie 3 by RT-qPCR of control (n=6-11) and OS (n=7-8) serum EV preparations. RT-qPCR was normalized against C. elegans external spike-in miR-39-3p RNA. White lines represent median. (b) Violin plots representing relative abundance of HSATI, HSATII, L1P1 and Charlie 3 DNA by qPCR, in the absence of reverse transcription, in control (n=12) and OS (n=8) serum EV preparations. qPCR was performed on equal proportions of nucleic acid extracted from 200 ul of OS and control serum. White lines represent median. (c) Diagnostic value of HSATI, HSATII, L1P1 and Charlie 3 DNA sequences in serum OS preparations. ROC curves were generated using data in (b). Groups were compared using two-tailed, unpaired, Mann Whitney U test; *P<0.05; **P<0.01; ***P<0.001.

FIGS. 4A-4C. FIG. 4: Repetitive Element Sensitivity to DNase in EV preparations. (a) Abundance of HSATI and HSATII sequences determined by qPCR in control (n=4) and OS (n=4) serum EV preparations isolated with PEG and either untreated (UT) or pretreated with DNase I or RNase A with or without NaCl prior to nucleic acid extraction. Treated groups were compared to untreated groups using unpaired Kruskal-Wallis test with uncorrected Dunn's test where each comparison stands alone; *, P<0.05. Error bars represent standard deviation of biological replicates. (b) Bioanalyzer electropherograms of equal proportions of nucleic acids prepared from 200 ul of representative control and OS EV preparations that were untreated or treated with DNase I or RNase A prior to nucleic acid extraction. (FU: fluorescence units). (c) Violin plots representing relative abundance of HSATII, L1P1 and Charlie 3 by RT-qPCR in control (n=4) and OS (n=4) serum EV preparations pre-treated with DNAse I. qPCR was normalized against C. elegans external spike-in miR-39-3p RNA. White lines represent median.

FIGS. 5A-5R. FIG. 5: Co-purification of OS-associated repetitive element DNAs with EVs in size exclusion chromatography but not exosome immunoaffinity capture. (a, b) Representative protein concentration (blue line) and EV concentration (black bars) elution profiles from control (a) and OS (b) serum separated by size exclusion chromatography (SEC). (c, d) Representative size distribution of control (c) and OS (d) serum EV particles in pooled fractions 6 and 7 analyzed by nanoparticle-tracking. (e, f) Relative abundance of HSATI and HSATII DNA in two control and two OS SEC (e) and PEG (f) EV fractions as defined by qPCR. (g, h) Representative protein concentration (blue line) and nucleic acid concentration (black bars) of pooled EV fraction (F6-7) and selected non-EV fractions (F8, F11, F12, F13 and F18) from the same representative control (g) and OS (h) SEC separations as in (a) and (b). (i, j) Abundance of HSATI (i) and HSATII (j) in pooled EV fractions 6-7 and non-EV fractions on one control and one OS SEC analysis as evaluated by qPCR. (k-n) SP-IRIS analyses by ExoView of EVs isolated by CD9-immunoaffinity capture. (k, 1) Representative concentration of control (k) and OS (1) fluorescent EV particles immunocaptured on the CD9, CD81 and CD63 antibody spots. Results depict the mean of the measurement of triplicate spots ±SEM, subtracted for IgG spot values and adjusted by dilution factor. (m, n) Representative size distribution of control (m) and OS (n) label-free EV particles immunocaptured on the CD9, CD81 and CD63 antibody spots. Results depict the mean of the measurement of triplicate spots ±SEM, subtracted for IgG spot values. (o, p) Size distribution and particle number of control (o) and OS (p) EVs isolated by CD9 immunoaffinity capture and analyzed by nanoparticle-tracking. (q, r) Violin plots representing abundance of HSATI (m) and HSATII (n) of control (n=6) and OS (n=8) immunoaffinity capture of CD9-positive exosomes evaluated by qPCR. White lines represent median. Groups were compared using two-tailed, unpaired, Mann Whitney U test; *P<0.05. Similar CD81 immunoaffinity capture results in FIG. 22.

FIGS. 6A-6B. Human satellite sequences not enriched in total cfDNA in OS patient sera. (a) Violin plots representing relative abundance of HSATI, HSATII, L1P1 and Charlie 3 by qPCR of control (n=11) and OS (n=7) whole serum. White lines represent median. (b) Diagnostic value of HSATI, HSATII, L1P1 and Charlie 3 in OS patient whole serum. ROC curves were generated using data in (a). Groups were compared using two-tailed, unpaired, Mann Whitney U test; *P<0.05.

FIGS. 7A-7D. Over-representation of RE vs. Non-RE DNAs in OS compared to control EVs. (a, b) Dot plots representing read counts (CPM) of HSATII (a) and L1 (b) in sequencing analyzed by TEtranscripts in control (n=12) and OS (n=12) serum EV preparations (with corresponding ROC curves below). c) Dot plots representing the mean CPM of 15 significantly over-represented RE sequences (left plots, with corresponding ROC curve for RE DNAs below) and 138 significantly under-represented non-RE sequences (right plots) in same samples as (a, b). d) Ratios of the base means of 15 over-represented REs to 138 under-represented non-RE sequences in same samples as (a, b) (with corresponding ROC curve below). In (a, b, d), groups were compared using two-tailed, unpaired, Mann Whitney U test and in (c), using Friedman followed by Dunn's multiple comparison tests; ***P<0.001; ****P<0.0001 (CPM: counts per millions).

FIG. 8. Increased levels of a 90 kDa protein in high density fractions of OS sera. 200 ul of serum from controls and OS patients were loaded on top of an iodixanol density gradient. 500 ng of combined fractions 10+11 were loaded on a bi-acrylamide gel and proteins revealed by silver staining.

FIGS. 9A-9B2. Workflow of the RE DNA and non-RE DNA assay. a) Sample preparation overview of simultaneous RNA and DNA library construction. b) Analysis of RE DNA proportions by next generation sequencing (b1) or by PCR product capture and probe hybridization of RE and non-RE DNA sequences.

FIGS. 10A-10D. Over-representation of RE DNAs in OS compared to control PEG- and gradient UC-EV preparations. Violin plots representing relative abundance of RE DNAs by qPCR in control (n=6) and OS (n=2) serum EV preparations form PEG-precipitation (a) and density gradient UC (c). (b and d) Diagnostic values of (a) and (c); respectively. Groups were compared using two-tailed, unpaired, Mann Whitney U test; **P<0.01; ***P<0.001; ****P<0.0001.

FIG. 11 Representation of repetitive elements in breast cancer compared to control EV serum preparations. Dot plots representing relative abundance of HSATI, HSATII, L1P1 and Charlie 3 by qPCR of control (n=10) and breast cancer patient (n=2) serum EV preparations. qPCR was performed on equal proportions of nucleic acid extracted from 50 ul of control, bladder and breast cancer patient serum.

FIG. 12. Differential representation of single copy nucleic acid sequences in OS versus control EV preparations. Volcano plot of the differentially represented features evaluated with DESeq2 between control and OS serum EV preparations. Single-copy genes represented significantly (FDR<0.05) and non-significantly (FDR>0.05) more than 2-fold in OS vs. control samples are shown in red and green, respectively. Those represented less than 2-fold in OS vs. control samples are shown in blue and grey.

FIGS. 13A-13C. Differential representation of repetitive element sequences in OS versus control EV preparations. (a-c) Tukey box plots with mean (vertical line), 25th-75th percentiles (boxes), and maximum and minimum values up to a 1.5× the interquartile range (whiskers) for OS (green) and control (red) sequencing libraries aligned to RepeatMasker. (a) Proportion of reads aligned to different repetitive element classes in OS and control EV preparations normalized to the sample with maximum abundance in the class. (b) Proportion of reads aligned to each repetitive element category. (c) Proportion of reads aligned to each LINE1 subfamily.

FIGS. 14A-14D. Over-representation of repetitive elements in OS EV-associated sequences. MA plot for the differentially represented sequences between control and OS serum EV preparations as defined by TEtranscripts using genome build hg19. (a) Differentially represented single-copy genes and repetitive elements (REs), significantly differentially represented in red. (b) Differentially represented single-copy genes, significantly differentially represented in red. (c) Differentially represented single-copy genes in black and REs in blue. (d) Differentially represented REs, significantly differentially represented in red. Arrow, the significantly over-represented HSATII. Arrowhead, significantly under-represented RE. Significantly differentially represented: FDR<0.05, Wald test.

FIGS. 15A-15D. Structure of repetitive elements examined by PCR. (a, left) HSATII gene composed of tandem repeats of 23 and 26 nucleotides. Blue and green arrows represent forward and reverse primers; respectively. (a, right) Electrophoresis gel of HSATII amplification in control and OS samples in duplicate. Arrows, bands of indicated sizes that were removed for sequencing. (b) L1P1 gene structure. Primers amplify a region of ORF2. Blue and green arrows represent forward and reverse primers; respectively, with their position indicated from the first nucleotide of ORF2. (c, d) Position of primers for HSATI and Charlie 3. Grey lines represent the amplicon for each repetitive element.

FIGS. 16A-16B. No differential representation of single-copy genes in OS compared to control EV preparations. (a) Violin plots of relative representation of HECDT2 by RT-qPCR in control (n=12) and OS (n=10) serum EV preparations. RTqPCR was normalized against C. elegans external spike-in miR-39-3p RNA added during nucleic acid extraction. White lines represent median. (b) Diagnostic value of HECDT2 in OS serum EV preparations. ROC curves were generated using data in (a). Groups were compared using two-tailed, unpaired, Mann Whitney U test; ns: P>0.05.

FIGS. 17A-17C. Over-representation of repetitive elements in OS compared to control EV preparations in a validation cohort. Re-evaluation of the results for the validation cohort in FIG. 3, omitting OS1 and OS3 samples also present in the discovery cohort.

FIGS. 18A-18B. Repeat element abundance relative to ethnicity and OS type. (a) Scatter plots representing relative abundance of HSATI, HSATII, L1P1 and Charlie 3 DNA similar to violin plots in FIG. 3C with each individual sample represented by colored dots according the OS sample source (CHLA and HLOH). (b) Scatter plots representing relative abundance of HSATI, HSATII, L1P1 and Charlie 3 DNA similar to violin plots in FIG. 3C with each individual sample represented by colored dots according to the OS type. Groups were compared using two-tailed, unpaired, Mann Whitney U test; *P<0.05; **P<0.01; ***P<0.001.

FIGS. 19A-19B. No differential abundance of single-copy genes in OS compared to control EV preparations. (a) Violin plots representing the relative abundance of ZNF3 and IL17RA DNA by TaqMan assay, in the absence of reverse transcription, in control (n=12) and OS (n=8) serum EV preparations. qPCR was performed on equal proportions of nucleic acid extracted from PEG precipitations of 200 ul of OS and control sera. White lines represent median. (b) Diagnostic value of ZNF3 and IL17RA in OS serum EV preparations. ROC curves were generated using data in (a). Groups were compared using two-tailed, unpaired, Mann Whitney U test; ns: P>0.05.

FIGS. 20A-20B. Abundance of repetitive elements DNAs co-purified with PEG precipitation and size exclusion chromatography (SEC) and normalized to particle concentration. Relative abundance of HSATI and HSATII DNA in two control and two OS SEC (a) and PEG (b) EV preparations was defined using the same qPCR reactions as shown in FIG. 5e-f (performed on equal proportions of nucleic acids extracted from PEG-precipitated or SEC-isolated EV preparations from 200 ul of OS and control sera), but with abundance normalized to the particle concentration of each sample.

FIGS. 21A-21P. Phenotyping of EVs isolated by PEG precipitation and CD9 immunoaffinity capture. SP-IRIS analyses by ExoView on control (top rows) and OS (bottom rows) samples obtained by PEG precipitation (a-c and i-k) and by CD9 immunocapture affinity (e-g and m-o). (a, e, i, m) Concentration of EV particles captured on the ExoView CD9, CD81, CD63, and CD41a antibody spots as measured by intrinsic fluorescence. Results depict the mean of the measurement of triplicate spots ±SEM, subtracted for IgG spot values and adjusted by dilution factor. (b, f, j, n) Representative size distribution of label-free EV particles immunocaptured on the CD9, CD81, CD63, and CD41a antibody spots. Results depict the mean of the measurement of triplicate spots ±SEM, subtracted for IgG spot values. (c, g, k, o) Immunophenotyping of EV particles on the CD9, CD81, CD63, and CD41a antibody spots determined using fluorescent antibodies. (d, h, 1, p) Control and OS EVs isolated by PEG precipitation (d and 1) and CD81 immunoaffinity-capture (h and p) and analyzed by nanoparticle-tracking. Panels e, m, f, and n are identical to FIGS. 5k, l, m, and n, respectively.

FIGS. 22A-22C. No co-purification of OS-associated repetitive element DNAs with EVs by CD81 immunoaffinity capture. (a, b) Size distribution and particle number of control (a) and OS (b) EVs isolated by CD81 immunoaffinity capture and analyzed by nanoparticle-tracking. (c) Violin plot representing abundance of HSATII of control (n=6) and OS (n=4) immunoaffinity capture of CD81-positive exosomes evaluated by qPCR. White lines represent median. Groups were compared using two tailed, unpaired, Mann Whitney U test; ns, not significant, p<0.05.

### DETAILED DESCRIPTION OF THE INVENTION

Provided herein are circulating biomarkers that distinguish OS patients from healthy controls, and a liquid biopsy for early OS detection and other cancers. Liquid biopsies may detect circulating tumor components including cfDNA, tumor cells, and extracellular vesicles (EVs) (14-1), a category that includes exosomes, shedding vesicles, microparticles, retroviral-like particles, ectosomes, microvesicles, oncosomes, and apoptotic bodies (20,21). EVs are released by most if not all cells (22) and carry components of their cell of origin such as proteins, lipids, metabolites, and various types of RNA (23,24). Among the different types of EVs, exosomes and oncosomes are more highly produced by cancer cells than by normal cells, are often present at increased levels at cancer diagnosis, may further increase during tumor progression (15), and carry cargo that reflects metastatic progression and treatment response (25,26). Moreover, EV preparations may contain exosomal as well as non-exosomal tumor components. Therefore, cancer biomarkers, such as OS biomarkers, in serum derived EV preparations were isolated.

To identify EV-associated OS biomarkers, the abundance of nucleic acid sequences in OS patient versus control serum EV preparations were compared. Specifically, small nucleic acids extracted from EV preparations were sequenced and examined for differential representation of unique as well as repetitive element sequences which are often produced and may be released by cancer cells (27), including by OS cells (28). Next it was evaluated whether the same sequences were differentially represented in different patient cohorts and by different EV isolation and analytic methods. Through these approaches circulating EV-associated repetitive element DNA sequences were identified that were more abundant in OS sera compared to healthy sera in two patient cohorts. Moreover, EV-associated repetitive element DNA sequences comprised an increased proportion of total sequences in the small nucleic acid preparations, and the ratio of certain repetitive element DNA sequences versus certain non-repetitive element sequences was increased.

### Definitions

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Any methods and materials similar or equivalent to those described herein can be used in the practice or testing of the present invention. Specific and preferred values listed below for radicals, substituents, and ranges are for illustration only; they do not exclude other defined values or other values within defined ranges for the radicals and substituents.

As used herein, the articles “a” and “an” refer to one or to more than one, i.e., to at least one, of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “reference” or “control” is meant a standard of comparison. For example, the marker level(s) present in a patient sample may be compared to the level of the marker in a corresponding healthy cell or tissue or in a diseased cell or tissue. As used herein, the term “sample” includes a biologic sample such as any tissue, cell, fluid, or other material derived from an organism.

As used herein a subject is any mammal, including humans, companion animals including cats and dogs, and livestock, including horses, pigs and cows.

The terms “treat,” “treating,” and “treatment,” as used herein, refer to therapeutic or preventative measures such as those described herein. The methods of “treatment” employ administration to a patient of a treatment regimen in order to prevent, cure, delay, reduce the severity of, or ameliorate one or more symptoms of the disease or disorder or recurring disease or disorder, or in order to prolong the survival of a patient beyond that expected in the absence of such treatment. Treatment for cancer includes active surveillance (during active surveillance, the tumor is monitored, and treatment would begin if it started causing any symptoms or problems or showed an alteration in the level of serum markers as described herein), surgery, radiation (such as external-beam radiation, including conventional radiation therapy, intensity modulated radiation therapy (IMRT)), 3-dimensional conformal radiation therapy; stereotactic radiosurgery, fractionated stereotactic radiation therapy or proton radiation therapy), immunotherapy and chemotherapy.

The term “effective amount,” as used herein, refers to that amount of an agent, which is sufficient to effect treatment, prognosis or diagnosis of cancer, when administered to a patient. A therapeutically effective amount will vary depending upon the patient and disease condition being treated, the weight and age of the patient, the severity of the disease condition, the manner of administration and the like, which can readily be determined by one of ordinary skill in the art.

Other terms used in the fields of recombinant nucleic acid technology, microbiology, immunology, antibody engineering and molecular and cell biology as used herein will be generally understood by one of ordinary skill in the applicable arts. Techniques and procedures may be generally performed according to conventional methods well known in the art and as described in various general and more specific references that are cited and discussed throughout the present specification. See, e.g., Sambrook et al., 2001, Molecular Cloning: A Laboratory Manual, 3rd ed., Cold Spring Harbor Laboratory Press, Cold Spring Harbor, N.Y., which is incorporated herein by reference for any purpose. Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

By “biologic sample” is meant any tissue, cell, fluid (such as blood or serum), or other material derived from an organism.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

As used herein the term “comprising,” “having” and “including” and the like are used in reference to compositions, methods, and respective component(s) thereof, that are present in a given embodiment, yet open to the inclusion of one more or more unspecified elements. The term “including” is used herein to mean, and is used interchangeably with, the phrase “including but not limited to.”

As used herein the term “consisting essentially of” refers to those elements required for a given embodiment. The term permits the presence of additional elements that do not materially affect the basic and novel or functional characteristic(s) of that embodiment of the invention. The term “consisting of” refers to compositions, methods, and respective components thereof as described herein, which are exclusive of any element not recited in that description of the embodiment.

### Assay/Method Steps/Components

The assays described herein provide numerous advantages. The RE DNA Quantitation assay requires a minimal volume of serum as low as 50 ul (or lower); it can conveniently be added to any blood test where serum or plasma is obtained at a screening exam. The entire procedure is as rapid as one day and cost-effective. Two variations of the assay that measure the proportion of repetitive element DNA sequences relative to total sequences involves additional steps yet has greater specificity.

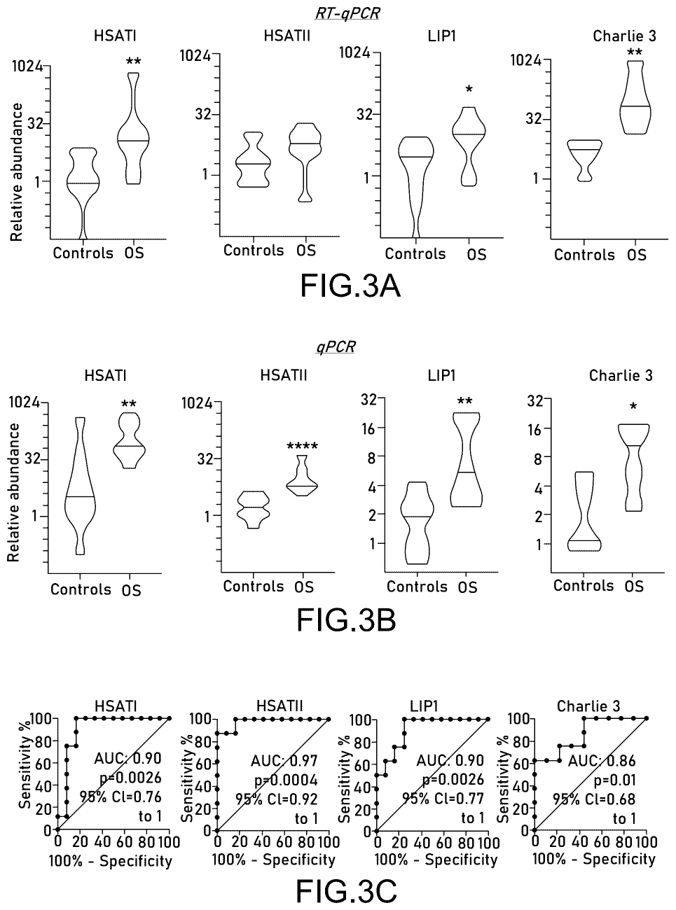

The RE DNA Quantitation assay provides high sensitivity and specificity as demonstrated by ROC curves (Cambier et al, FIG. 3c) and it is the only test for early diagnosis of osteosarcoma as liquid biopsy based on quantitation of repetitive element DNA fragments. Other osteosarcoma detection blood tests may have been proposed, particularly to detect osteosarcoma-related microRNAs, but they are not as specific or as sensitive, and they require more preparation steps. Whereas this RE DNA Quantitation Test requires only PEG precipitation, nucleic acid isolation, and qPCR (miRNA tests require adaptor ligation, and reverse transcription of the miRNA to produce a cDNA, and then qPCR of the cDNA). Those other assays also require greater serum or plasma volumes than the 50 ul that suffices for the instant assays.

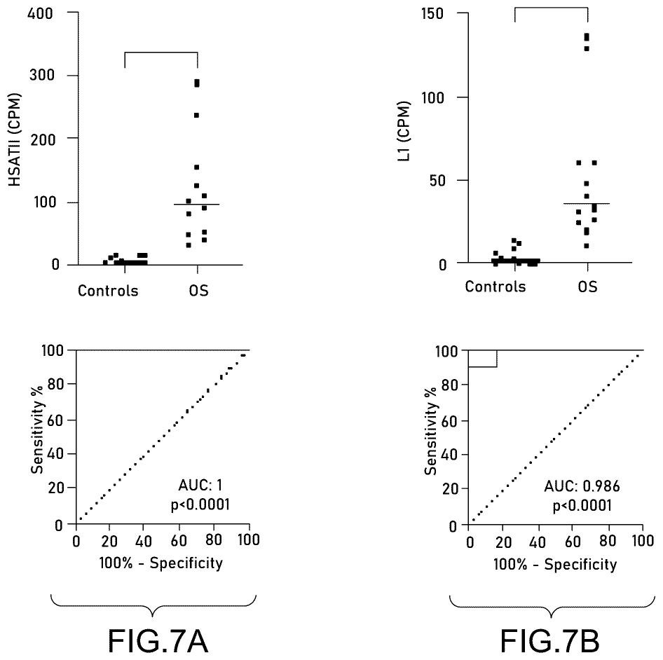

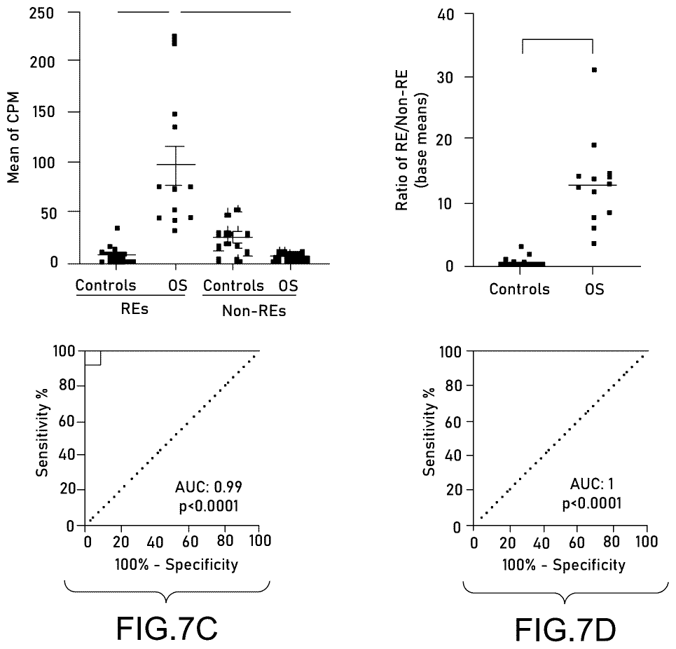

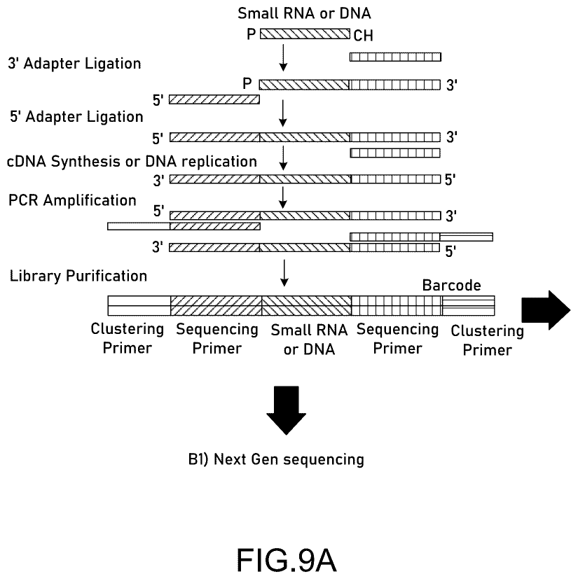

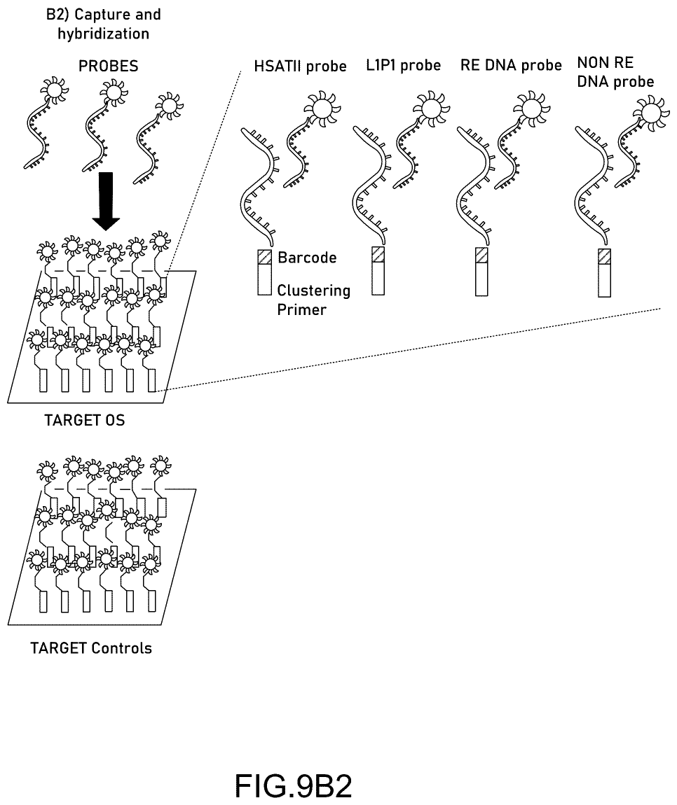

A higher sensitivity version of the RE DNA assay measures the proportion of RE DNA relative to total sequences in the same nucleic acid preparation. The high sensitivity of this RE DNA Proportion Test is shown in (FIG. 7 panel a-c). This high sensitivity “RE DNA Proportion Test,” test requires adaptor ligation to all of the isolated genomic DNA (gDNA) and RNA (including RE and non-RE sequences), reverse transcription of the RNA to produce a cDNA, and then PCR amplification of all gDNA and cDNA(s) and either sequencing or a hybridization-based method to detect the RE DNA proportion (FIG. 9) (or other established approaches to measuring proportions). Although such preparation steps are similar to miRNA assays, they have the crucial difference that RE DNA and cDNA are co-amplified (rather than cDNA only) and measurement of RE DNA is critical. Despite the additional steps, the assay may be performed on as little as 50 ul of serum on account of the very high RE DNA abundance.

In another iteration, the ratio of proportions of certain RE DNA and certain non-RE sequences is evaluated by a) defining the proportions of such sequences as described for the RE DNA Proportion Test (above) and then b) defining the ratio of these proportions. The higher sensitivity of this RE DNA Ratio Test results from the more consistently increased ratio of RE DNA and decreased levels of certain non-RE RNAs (FIG. 7d) when compared to the measurement of increased proportion of RE DNA alone (FIG. 7c (left).

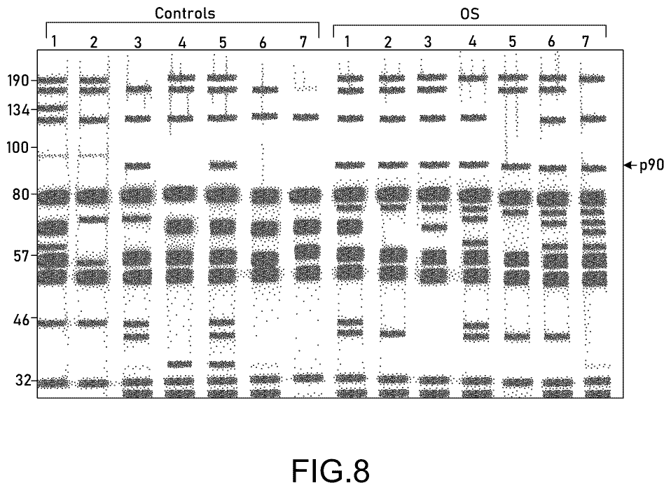

Each of the above RE DNA assays may be used in combination with assays of other OS serum markers to strengthen the diagnostic sensitivity, such as the recently discovered and reproducibly increased p90 protein in density gradient EV fractions (FIG. 8) and extracellular vesicle and particle (EVP) proteins that are common to many cancers (e.g., VCAN, TNC, and THBS2) or preferentially detected in different tumor types (e.g., ACTA1, ACTG2, ADAMTS13, HGFAC, MME, and TNC in osteosarcoma) (Hoshino et al, Cell. Volume 182, Issue 4, 20 Aug. 2020, Pages 1044-1061.e18; doi.org/10.1016/j.cell.2020.07.009).

The tests provided herein quantitate the abundance or proportional representation of specific repetitive elements DNAs that are co-purified with EV preparations from a small volume of serum (e.g., 50 ul). The steps include:

1. Serum or plasma isolation. Serum is obtained in serum separator tubes and separated from cells or clot by standard methods. Plasma (blood drawn in EDTA tubes, then separated from cells) might also be used if plasma is converted back to a serum-like state.

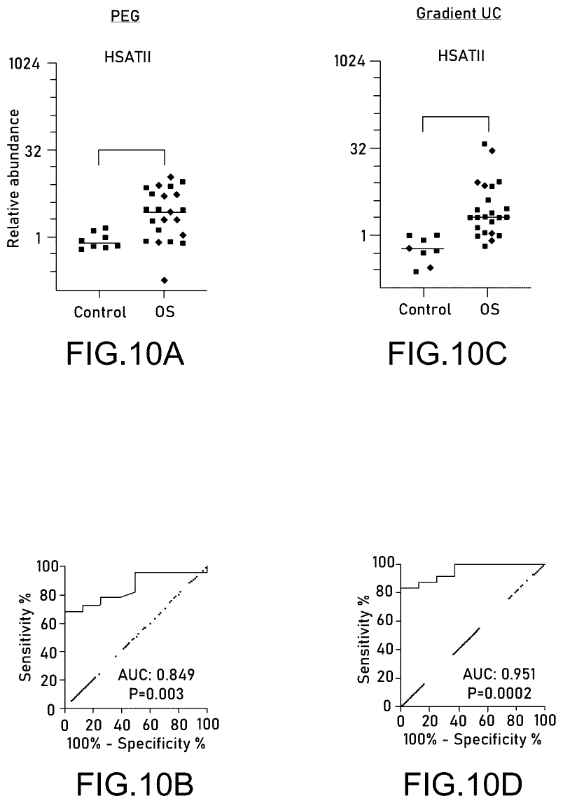

2. EV preparation. A standardized amount of serum (e.g., 50 ul) is subjected to either a) polyethylene glycol (PEG) precipitation (Rider et al. Sci Rep2016 Apr. 12; 6:23978. doi: 10.1038/srep23978.), b) size exclusion chromatography (SEC) (Nordin et al. Nanomedicine. 2015 May; 11(4):879-83. doi: 10.1016/j.nano.2015.01.003. Epub 2015 Feb. 4.), or c) density gradient centrifugation (FIG. 10), or other separation methods that are based on the same physical/chemical principles. All methods enrich for EVs and non-EV components (PMID: Andreu et al. J Extracell Vesicles. 2016 Jun. 20; 5:31655. doi: 10.3402/jev.v5.31655. eCollection 2016 and Brennan et al. Sci Rep. 2020 Jan. 23; 10(1):1039. doi: 10.1038/s41598-020-57497-7.). Of note, density gradient centrifugation (or other separation methods based on density) may be used to improve detection of the increased EV-associated RE DNAs in serum of OS (and other cancer) patients.

3. Small nucleic acid isolation. Nucleic acids are extracted from PEG precipitations, from SEC void volumes, or from density gradient EV fractions using commercial small RNA enrichment kits (sera-MiR (SBI) or miRNeasy micro (Qiagen)). This is a novel step for assay of a DNA species and preferentially isolates osteosarcoma and other cancer related RE DNAs.

4. The RE DNA Quantitation Test. A proportion of nucleic acids isolated from osteosarcoma (or other cancer) and control PEG, SEC, or density gradient preparations is subjected to qPCR (using primers determined by PCR primer-design programs, reported in the literature, or otherwise designed by the inventors) to quantitate the abundance of specific RE DNA sequences, and comparison is made between osteosarcoma (or other cancer) and control reference samples.

5. The RE DNA Proportion Test. A proportion of nucleic acids isolated from osteosarcoma (or other cancer) and control PEG, SEC, or density gradient preparations is subjected to adaptor ligation (using a T4 RNA ligase-like enzyme that ligates adaptors to the 5′ and 3′ ends of DNA as well as RNA), reverse transcription (using primers that are complementary to the adaptors), and PCR-based amplification (using PCR primers that are complementary to the ligated adaptors), and determination of the proportional representation of RE sequences of interest either a) by massively parallel next generation sequencing or b) by hybridization-based capture of PCR product to immobilized probes followed by hybridization of fluorescent probes to the captured RE sequences of interest. Other available approaches to measure the proportions may also be used.

6. The RE DNA Ratio Test. A proportion of nucleic acids isolated from osteosarcoma (or other cancer) and amplified as in (5), followed by determination of the proportional representation of both RE sequences of interest and non-RE sequences of interest (by massively parallel next generation sequencing or hybridization-based capture of PCR product and secondary hybridization as in (5)), and calculation of the ratio of the RE and non-RE sequences.

Also provided herein are kits for EV-associated DNA and non-RE nucleic acid enrichment, wherein said kit comprising components including sample gathering, EV isolation/preparation components and/or components to quantitate RE DNA and non-RE sequences, including chips that may be used to capture PCR products and devices to read the fluorescent signals based on secondary hybridization, along with instructions for use and optionally a control sample.

### Cancer

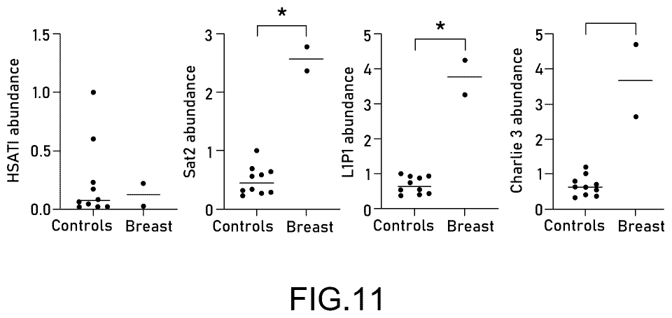

As EV-associated RE DNAs were shown to be elevated in two very different tumors affecting very different patient populations (osteosarcoma and breast cancer (see, for example, FIG. 11)) the RE DNA test may further be useful for early detection or monitoring therapy response and relapse for other cancers including solid cancers such as: Central Nervous System Cancers, Adult Ocular and Orbital (Ocular Adnexa) Tumors, Head and Neck Cancer, Thyroid Cancer, Endocrine and Neuroendocrine Tumors, Breast Cancer, Lung Cancer, Esophageal Cancer, Hepatocellular Carcinoma, Pancreatic Cancer, Biliary Tract Cancer, Gastric Cancer, Bladder Cancer, Prostate Cancer, Colorectal Cancer, Anal Cancer, Germ-Cell Cancer of the Testis and Related Neoplasms, Renal Cell Cancer, Ovarian, Fallopian Tube, and Primary Peritoneal Cancer, Uterine Cancer, Cervical Cancer, Carcinoma of the Vagina and Vulva, Gestational Trophoblastic Neoplasia, Non-Melanoma Skin Cancer, Malignant Melanoma, Primary Bone Tumors, Soft Tissue Sarcomas; and blood cancers such as Leukemias, Hodgkin Lymphoma, Non-Hodgkin Lymphoma, Multiple Myeloma; and/or pediatric cancers, including solid tumors such as bone and soft tissue sarcomas, kidney, liver, eye cancers; neuronal cancers such as neuroblastoma and brain or eye cancers, blood cancers such as B and T-cell leukemias and myeloid leukemias.

The assay described herein, validated for osteosarcoma, would serve for cancer screening in genetically predisposed children, like retinoblastoma, Li-Fraumeni, Bloom, Werner and Rothmund-Thomson syndromes, children exposed to radiation or alkylating agents, Diamond-Blackfan anaemia syndrome and in adults with bone disorders such as Paget's Disease patients (use in osteosarcoma predisposition syndromes) and at risk for breast or ovarian cancer due to inherited mutations (such as BRCA1 or BRCA2).

As osteosarcoma is characterized by high chromosomal instability, there is no predominant tumor suppressors or oncogenes to focus on as biomarkers. The discovery of specific repetitive element DNAs bypasses the need for gene-specific biomarkers. Additionally, the assay/diagnostic test is a unique and inexpensive assay for relapse of osteosarcoma and other cancers.

### Treatment

The terms “treat,” “treating,” and “treatment,” as used herein, refer to therapeutic or preventative measures such as those described herein. The methods of “treatment” employ administration to a patient of a treatment regimen in order to prevent, cure, delay, reduce the severity of, or ameliorate one or more symptoms of the disease or disorder or recurring disease or disorder, or in order to prolong the survival of a patient beyond that expected in the absence of such treatment. Treatment for a cancer includes active surveillance (during active surveillance, the tumor is monitored, and treatment would begin if it started causing any symptoms or problems or showed an alteration in the level of markers as described herein), surgery, radiation (such as external-beam radiation, including conventional radiation therapy, intensity modulated radiation therapy (IMRT), 3-dimensional conformal radiation therapy; stereotactic radiosurgery, fractionated stereotactic radiation therapy or proton radiation therapy), immunotherapy and chemotherapy.

The term “effective amount,” as used herein, refers to that amount of an agent, which is sufficient to effect treatment, prognosis or diagnosis of cancer, when administered to a patient. A therapeutically effective amount will vary depending upon the patient and disease condition being treated, the weight and age of the patient, the severity of the disease condition, the manner of administration and the like, which can readily be determined by one of ordinary skill in the art.

The early detection of malignancy based on the RE DNA test is specifically herein linked to treatment with appropriate modalities, which may include Surgery, Chemotherapy (e.g., with Methotrexate, Doxorubicin, Cisplatin or carboplatin, Ifosfamide, Cyclophosphamide, Etoposide, Gemcitabine), Radiation therapy, Bone marrow transplant, Immunotherapy, Hormone therapy, Targeted drug therapy, Cryoablation, or Radiofrequency ablation. An example is the treatment of triple negative breast cancer as well as other cancers (Byrum, A. K., Vindigni, A. & Mosammaparast, N. Defining and Modulating ‘BRCAness’. Trends Cell Biol 29, 740-751, doi:10.1016/j.tcb.2019.06.005 (2019), incorporated herein by reference) (e.g., prostate (Wedge, D. C. et al. Sequencing of prostate cancers identifies new cancer genes, routes of progression and drug targets. Nat Genet 50, 682-692, doi:10.1038/s41588-018-0086-z (2018), incorporated by reference), colon (Yaeger, R. et al. Clinical Sequencing Defines the Genomic Landscape of Metastatic Colorectal Cancer. Cancer Cell 33, 125-136 e123, doi:10.1016/j.ccell.2017.12.004 (2018); incorporated by reference), and pancreatic adenocarcinoma (Cancer Genome Atlas Research Network. Electronic address, a. a. d. h. e. & Cancer Genome Atlas Research, N. Integrated Genomic Characterization of Pancreatic Ductal Adenocarcinoma. Cancer Cell 32, 185-203.e113, doi:10.1016/j.ccell.2017.07.007 (2017), incorporated by reference), Ewing's sarcoma (Brenner, J. C. et al. PARP-1 inhibition as a targeted strategy to treat Ewing's sarcoma. Cancer Res 72, 1608-1613, doi:10.1158/0008-5472.CAN-11-3648 (2012); incorporated by reference) that often have a high degree of BRCAness (representing a defect in homologous recombination repair, due to or mimicking BRCA1 or BRCA2 loss, or in replication fork protection (RFP), with increased genomic instability) with a combination of DNA damaging chemotherapy (such as with platinum based compounds) or radiotherapy combined with PARP inhibitors (Byrum, A. K., Vindigni, A. & Mosammaparast, N. Defining and Modulating ‘BRCAness’. Trends Cell Biol 29, 740-751, doi:10.1016/j.tcb.2019.06.005 (2019)). As the two cancers already identified as having increased serum EV-associated RE DNA (osteosarcoma and triple negative breast cancer) both have high levels of BRCAness, the unique combination of RE DNA screening and BRCAness-directed therapies is an embodiment of the invention.

The following example is intended to further illustrate certain particularly preferred embodiments of the invention and are not intended to limit the scope of the invention in any way.

### Example

### Materials and Methods

Patients and Samples

This study was reviewed and approved by the institutional review board at Children's

Hospital Los Angeles (approval no. CCI-13-00223) and at Henan Luoyang Orthopedic Hospital (approval no. 2015-01). All participants gave a written informed consent. Parents/Legally authorized persons gave informed consent on behalf of all minors and subjects above 14 years old gave assent. All analyses were conducted in accordance with relevant guidelines and regulations. Blood samples were collected during a clinically indicated venipuncture from previously untreated patients with primary diagnosis of OS and from volunteer subjects with no known medical conditions, i.e. healthy controls. Control sera for the validation cohort were obtained from local volunteer subjects and from Innovative Research Inc. (Novi, MI, USA). Blood was drawn in serum separator collection tubes (SST), clotting was allowed for 30 min at room temperature in vertical position and then tubes were centrifuged at 1,000 g for 10 min at 4° C. Serum was collected, immediately aliquoted, and stored at −80° C.

Discovery Cohort

EV isolation, nucleic acid extraction, and sequencing. Serum EVs were isolated using ExoQuick (System Biosciences Inc. (SBI), Mountain View California, USA) and aliquots frozen. One aliquot was used for NTA analyses and on confirmation of high EV purity aliquots were thawed and nucleic acid extracted using SeraMir (SBI) without DNase treatment, according to manufacturer instructions. The sequencing library was constructed using TailorMix miRNA Sample Preparation (SeqMatic) with a selection of small nucleic acids from 140 to 300 bases. 5′-RNA adapters and 3′-DNA adapters (SeqMatic, personal communication) were directly ligated to nucleic acid substrates, followed by PCR amplification. Libraries were sequenced to generate single-end 50 bp reads on MiSeq 500 platform (Illumina).

Validation Cohort

EV isolation and nucleic acid extraction. Serum was cleared by centrifugation at 3,000×g for 15 min at 4° C. For polyethylene glycol (PEG) precipitation, 50-200 ul of cleared serum was combined with an equal volume of freshly prepared 16% PEG 6000 (Sigma-Aldrich) in 1M NaCl, to give a final concentration of 8%, incubated for 30 min on ice, centrifuged in a tabletop microfuge at 16,000×g for 2 min at room temperature (Eppendorf, model 5424 R using an FA-45-24-11 fixed angle rotor) and the pellet resuspended in a volume of PBS equal to that of the starting serum volume. For size exclusion chromatography (SEC), ˜300 ul of cleared supernatant was centrifuged at for 30 min at 4° C. in a fixed angle rotor and loaded onto a glass Econo-column (BioRad, 10 cm height, 1.5 cm diameter) packed with Sephacryl S-300 High Resolution (GE Healthcare) and pre-washed with 0.32% Sodium Citrate in PBS. The cleared serum was allowed to enter the resin by gravity flow and eluate collected in 20 fractions of 15 drops (˜500 ul) on a Model 2110 Fraction Collector (BioRad). For each fraction, the protein concentration and the presence of EVs was characterized by Bradford method (BioRad) and nanoparticle tracking analysis (see below), respectively. EV fractions were concentrated on a 100 kDa Amicon ultra centrifugal filter (Millipore) from 2×500 μl to a final volume of ˜100 ul. Immunoaffinity capture of CD81+ or CD9+ EVs was carried out using the Exo-FLOW™ Exosomes Purification Kit (SBI, Mountain View California). Briefly, 200 ul of cleared serum was precipitated with 200 ul of 16% PEG 6000 as above, and the pellet re-suspended in 200 ul of PBS. 50 ul of this EV preparation were incubated in 20 μl of anti-CD81 or anti-CD9 pre-coated magnetic beads (9.1 μm) on a rotating rack at 4° C. overnight. CD81+ or CD9+ EVs were eluted from the beads in the Exosome Elution Buffer at 25° C. for 30 min.

Nucleic acids were extracted from 20 to 200 ul of EV preparations (or from 50 ul of serum) using miRNeasy Micro kit (Qiagen) and suspended in 14 ul of RNase/DNasefree H₂O (depending on the initial volumes of serum) according to the manufacturer's instruction. For samples intended for reverse-transcription, a spike-in control (C. elegans miR-39-3p) miRNA mimic (Qiagen) was added (1.6×10⁹ copies) after the lysis step. Nucleic acid size and concentration were analyzed on an RNA Pico 6000 chip using an Agilent Bioanalyzer (Agilent, Palo Alto, CA, USA), equipped with Expert 2100 software, which generated an electrophoretic profile and the corresponding ‘pseudo’ gel of the sample. After separation, nucleic acid sizes were normalized to a 25 bp RNA marker. Samples showing nucleic acids of >200 bp were eliminated from the study.

Particle Size and Concentration Measurement by Nanoparticle Tracking Analysis

EV preparations were analyzed by nanoparticle tracking using a NanoSight NS300 (Malvern, Worcestershire, U.K.) configured with a high sensitivity sCMOS camera (OrcaFlash2.8, Hamamatsu C11440, NanoSight Ltd). In brief, each sample was mixed by vortexing, and subsequently diluted in particle-free PBS to obtain a concentration within the recommended measurement range (10⁸-10⁹ particles/mL), corresponding to dilutions from 1:100 to 1:500. After optimization, settings were kept constant between measurements. Ambient temperature was recorded manually and did not exceed 25° C. Approximately 20-40 particles were in the field of view for each measurement. Three videos of 30 s duration were recorded for each sample. Experiment videos were analyzed using NTA 3.2 Dev Build 3.2.16 software (Malvern).

Single Particle Interferometric Reflectance Imaging Sensing (SP-IRIS)

EVs from PEG and immunocapture preparations were analyzed on ExoView R100 platform (Nanoview Biosciences, MA). Briefly, EVs within these preparations were immunocaptured on a multiplexed microarray chip with CD9, CD81 CD63, and CD41a antibody spots, as well as negative control IgG antibody spots to determine the level of non-specific binding, and then probed for CD9, CD81, CD63, and CD41a surface markers with respective additional fluorescent antibodies. EVs from PEG preparation and eluted EVs from immunoaffinity were diluted in solution A (Nanoview Biosciences, MA). The samples were incubated on the ExoView Tetraspanin Chip (EV-TC-TTS-01) placed in a sealed 24-well plate for 16 h at room temperature. The chips were then washed three times in 1 ml PBST for 3 min each on an orbital shaker. Then, chips were incubated with ExoView Tetraspanin Labeling ABs (EV-TC-AB-01) that consist of anti-CD81 Alexa-555, anti-CD63 Alexa-488, and anti-CD9 Alexa-647. The antibodies were diluted 1:5000 in PBST with 2% BSA. The chips were incubated with 250 μL of the labeling solution for 2 h. The chips were then washed once in PBST, three times in PBS followed by a rinse in filtered deionized water and dried. Immunocaptured EVs on the microarray chip were imaged on a single EV-basis with the ExoView R100 reader using the nScan2 2.9 acquisition software. The data were then analyzed using the NanoViewer 2.9 software (Nanoview Biosciences, MA) that counts and sizes fluorescent nanoparticles immunocaptured on the antibody spots. For exosome analysis the size window was selected to include particle sizes from 50-200 nm.

Reverse Transcription (RT) and qPCR

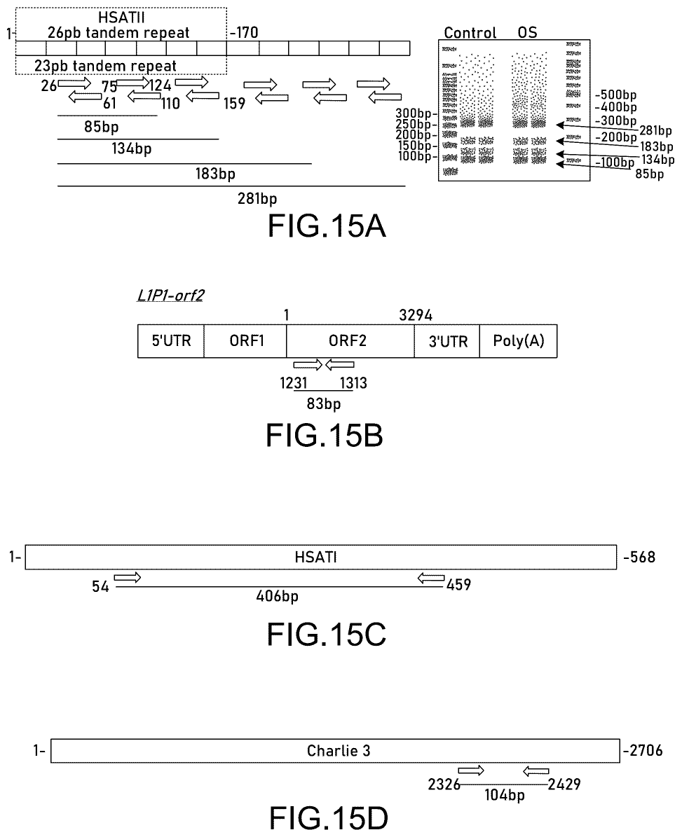

Equal volumes of nucleic acid prepared as above were reverse transcribed using iScript™ cDNA Synthesis Kit (Bio-Rad) in 20 ul volume according to the manufacturer's protocol. 0.5 ul of the samples produced with or without the RT step were analyzed in 10 ul qPCR reactions with iQ™ Green Supermix (Bio-Rad) on an ABI 7900 Fast Real-Time PCR System (Applied Biosystems) with the following cycling parameters: 94° C., 30 sec; 59° C., 15 sec; 68° C., 25 sec for 35 cycles. Relative sequence abundance was determined by the ΔΔCt method. In most PCR runs, a negative control with no nucleic acid template was added and never generated PCR product. PCR primers were designed manually to have a melting temperature of 58° C. and to generate amplicons of ˜100 bp or as previously described for HSATI (62) and HSATII (37) (FIG. 15) and obtained from Integrated DNA Technologies:

HSATI F:
(SEQ ID NO: 1)
5′-TAATGTGTGGGCTTGGGATT-3′,
HSATI R:
(SEQ ID NO: 2)
5′-TGCATATGGAAAATACAGAGGCTA-3′
(amplicon: 406 bp);
HSATII F:
(SEQ ID NO: 3)
5′-ATTCGATTCCATTCGATGATGATTCC-3′,
HSATII R:
(SEQ ID NO: 4)
5′-GGAACCGAATGAATCCTCATTGAATG-3′
(prominent amplicons; 85, 134, 183 and
281 bp);
L1P1-orf2 F:
(SEQ ID NO: 5)
5′-ATCAGAGAATACTACAAACACCTCTAC-3′,
L1P1-orf2 R:
(SEQ ID NO: 6)
5′-AGAGTGTATGTGTCGAGGAAT-3′
(amplicon: 83 bp);
Charlie 3 F:
(SEQ ID NO: 7)
5′-ACAAAAGCACTGAAAAGCCTGC-3′;
Charlie 3 R:
(SEQ ID NO: 8)
5′-TCCAGTCTACTCCGTAATCTCGT-3′
(amplicon: 104 bp);
HECDT2 F:
(SEQ ID NO: 9)
5′-TGTGAAAGACTTTCAGGAAGATGTAGAAAAA-3′,
HECDT2 R:
(SEQ ID NO: 10)
5′-GAGGGAGTGGCATCTTTCTTAAATG-3′
(amplicon: 131 bp).
For ZNF3 and IL17RA, TaqMan primers were used
(IL17RA: Hs01285262_cn and ZNF3: Hs03631848_cn
(Applied Biosystems) and PCR reactions were
performed according to the manufacturer′s
instructions.

DNase I and RNase A Treatment of EV Preparations

Intact EV preparations were treated with DNase I (Qiagen) in RDD buffer for 15 min at room temperature and then inactivated for 10 min at 70° C. Intact EV preparations were treated with RNase A (Thermo Scientific) at final concentration 0.4 ug/ul with or without NaCl at final concentration 1 M for 10 min at 37° C. 39 and inactivated by RNase inhibitor (Takara) at final concentration 2 u/ul.

RNA-Seq Data Processing, Alignment and Analysis

Fastq files were aligned to GRch38 (for analysis 1) or hg19 (for analysis 2) using STAR using the parameters recommended for TEtranscripts (i.e., allowing for up to 100 alignments per read) (34), and the resulting BAM files were processed using TEtranscripts to quantify both non-repetitive element and repetitive element abundance.

Statistical Analysis

Groups were compared using two-tailed, unpaired, Mann Whitney U test (*P<0.05, **P<0.01, ***P<0.001, ****P<0.0001). All analyses were performed using Prism 8 software (GraphPad).

### Results

### Over-Representation of Repetitive Element Sequences in OS EV Preparations

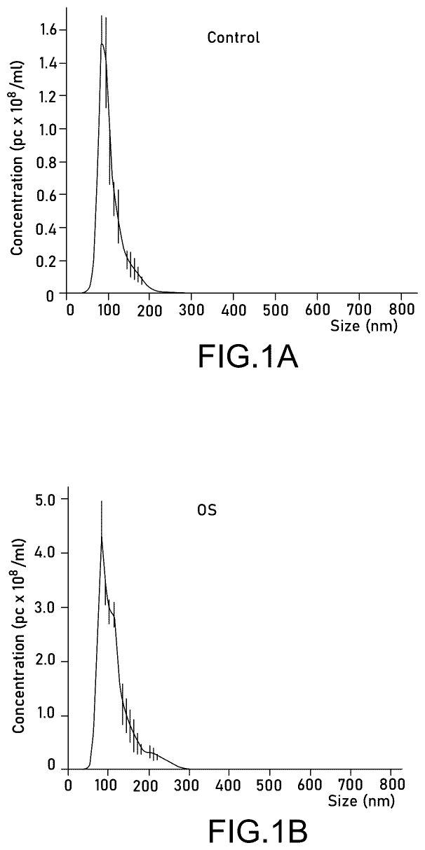

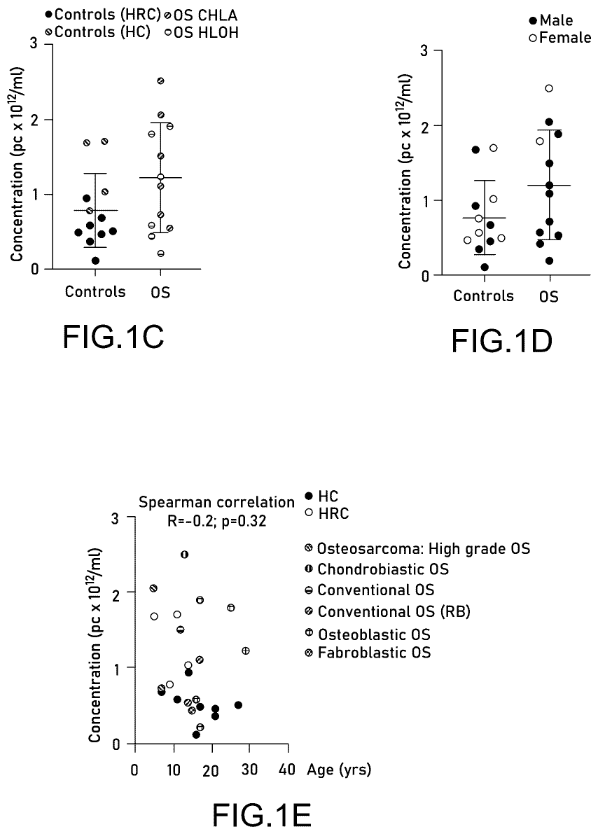

To identify OS biomarkers, nucleic acid sequences associated with EV preparations from sera of OS patients and healthy controls were compared. Initial analyses were performed on a discovery cohort of treatment-naïve OS patients from Children's Hospital Los Angeles (CHLA) and Henan Luoyang Orthopedic Hospital (HLOH), comprised of males and females between 5 and 29 years old and presenting with different OS types. Control cohorts were comprised of healthy siblings of hereditary retinoblastoma patients who had not developed retinoblastoma (hereditary retinoblastoma controls; HRCs) and unrelated approximately age-matched healthy individuals (healthy controls; HC) (Table 1). EV preparations were made with the commercial ExoQuick kit based on polyethylene glycol (PEG) precipitation, with recognition that EV as well as non-EV components are isolated (29,30). Nanoparticle tracking analysis of each sample revealed similar size distributions of control and OS EVs between 50 and 150 nm, which is characteristic of exosomes (FIGS. 1A and B). EV concentrations were not significantly higher in sera from OS patients compared to controls (FIG. 1C). Likewise, EV concentrations were similar in OS patient serum from USA (CHLA) and China (HLOH) and for different patient ages, genders, and OS types (FIGS. 1C-E).

TABLE 1
Osteosarcoma patient and control donor characteristics.
a. Discovery cohort (Nucleic acid sequencing)

| | | | | |
|---|---|---|---|---|
| Source1 | Sample2 | Gender | Age (yrs) | OS type |

Osteosarcomas

| | | | | |
|---|---|---|---|---|
| CHLA | OS-C1 | M | 7 | High grade, extensive necrosis |
| | OS-C2 | F | 13 | Chondroblastic, necrosis |
| | OS-C3 | 2 | 12 | Conventional |
| | OS-C4 | V | 5 | High grade, small cell variant |
| | OS-C7 | M | 17 | Conventional secondary to RB |
| | OS-C8 | M | 14 | Conventional secondary to RB |
| HLOH | OS-H6 | M | 16 | Osteoblastic |
| | OS-H7 | M | 17 | Osteoblastic |
| | OS-H11 | F | 25 | Osteoblastic |
| | OS-H19 | M | 15 | Fibroblastic |
| | OS-H20 | M | 17 | Osteoblastic |
| | OS-H27 | M | 29 | Osteoblastic |

Controls

| | | | |
|---|---|---|---|
| CHLA | HC1 | F | 17 |
| | HRC1 | M | 5 |
| | HRC2 | F | 14 |
| | HRC3 | F | 11 |
| | HRC4 | F | 9 |
| HLOH | HH12 | M | 10 |
| | HH17 | M | 14 |
| | HH21 | M | 21 |
| | HH23 | M | 16 |
| | HH25 | M | 27 |
| | HH28 | F | 21 |
| | HH29 | F | 11 |

b. Validation cohort (qPCR)

| | | | | |
|---|---|---|---|---|
| Source1 | Sample2 | Gender | Age (yrs) | OS type |

Osteosarcomas

| | | | | |
|---|---|---|---|---|
| CHLA | OS-C1 | M | 7 | High grade, extensive necrosis |
| | OS-C3 | F | 12 | Conventional |
| | OS-C5 | M | 13 | Conventional |
| | OS-C9 | M | 19 | Conventional |
| HLOH | OS-H21 | M | 43 | High grade, extensive necrosis |
| | OS-H24 | M | 46 | Osteoblastic |
| | OS-H25 | F | 16 | Osteoblastic |
| | OS-H34 | M | 14 | Osteoblastic |

Controls

| | | | |
|---|---|---|---|
| CHLA | HC2 | F | 38 |
| | HC3 | M | 38 |
| IR | HI1 | M | 20 |
| | HI2 | M | 19 |
| | HI3 | F | 20 |
| | HI4 | F | 18 |
| | HI5 | M | 20 |
| | HI6 | M | 19 |
| | HI7 | M | 20 |
| | H8 | F | 18 |
| | HI9 | F | 19 |
| | HI10 | F | 20 |

Abbreviations: OS-C, osteosarcoma from CHLA; OS-H, osteosarcoma from HLOH; HC, healthy control from CHLA; HH, Healthy control from HLOH; HI, healthy control from Innovative Research Inc.; HRC, hereditary retinoblastoma sibling control.

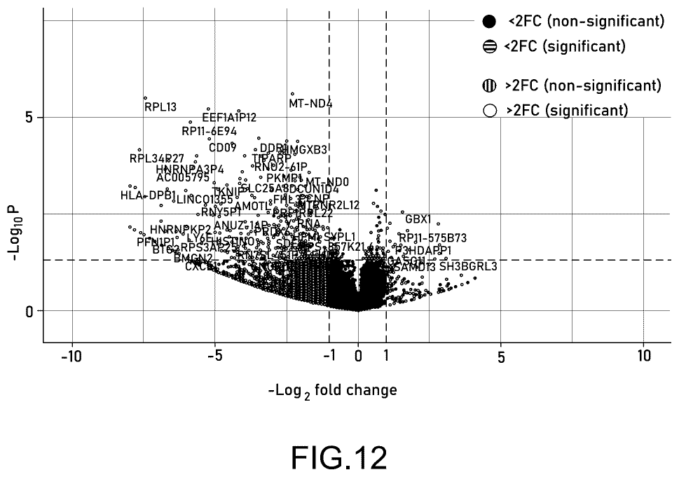

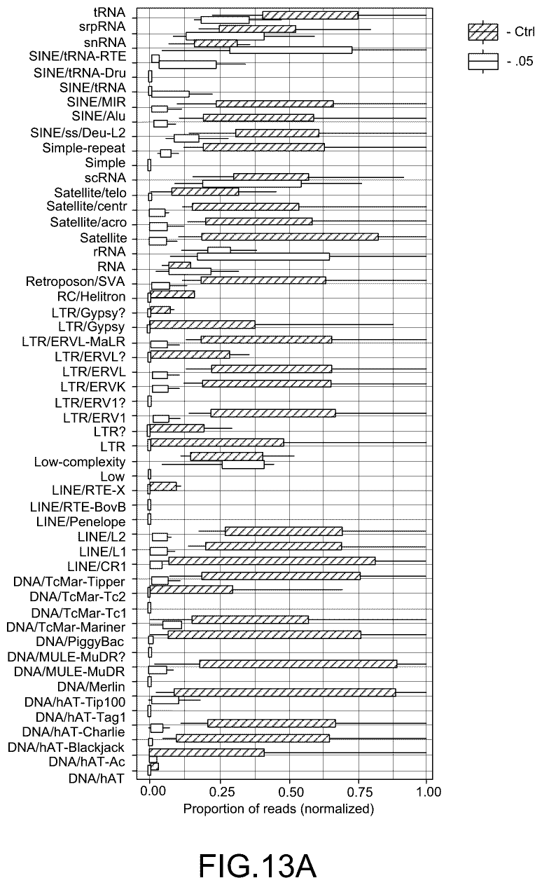

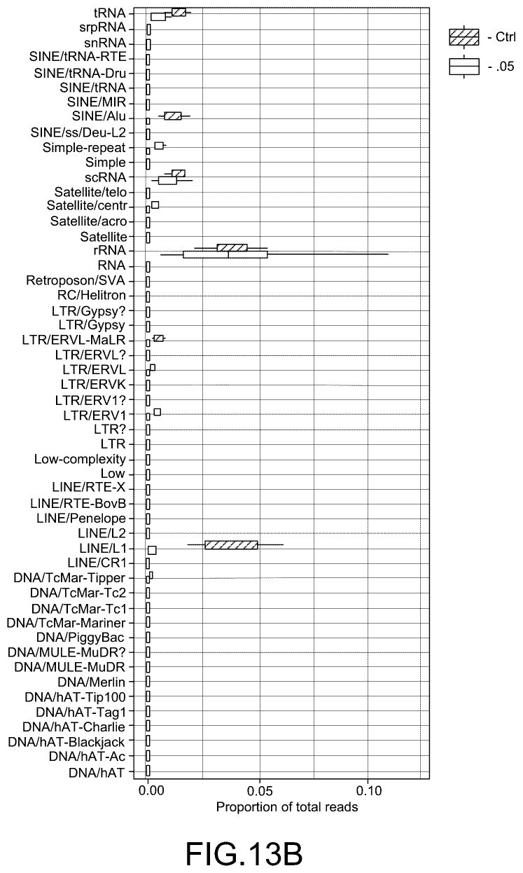

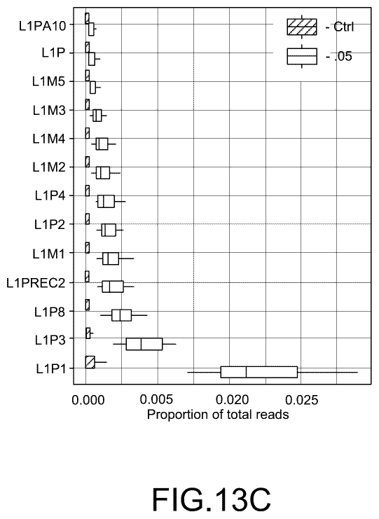

To detect differentially represented EV-associated RNA and DNA sequences, nucleic acids were extracted from OS and control EV preparations using SeraMir small RNA enrichment kit (SBI) without DNase treatment and a sequencing library was built by addition of a 5′-RNA adapter and a 3′-DNA adapter followed by PCR amplification and sequencing. Comparison of uniquely mapped sequences using DESeq2 (31) identified 107 significantly over-represented genes and 587 significantly under-represented genes (>2-fold change, p.adj<0.05) in OS samples (FIG. 12). However, the overrepresented sequences had poor OS sensitivity and specificity (not shown). As a far greater proportion of genes were under-represented in OS samples, it was considered whether the analysis of uniquely mapped sequences ignored potentially relevant overrepresented non-uniquely mapped sequences. Thus, the differential representation of the major repetitive element categories was evaluated after aligning reads to RepeatMasker (32). This indicated that OS serum EV preparations had greater representation of almost all repetitive element categories including the most abundant LINE1, LTR/ERV, a-satellite and SINE/Alu categories but not the low complexity ribosomal RNA and scRNA sequences (FIGS. 13A, B). The most highly represented sequence was the LINE1 family member L1P1 (FIG. 13C).

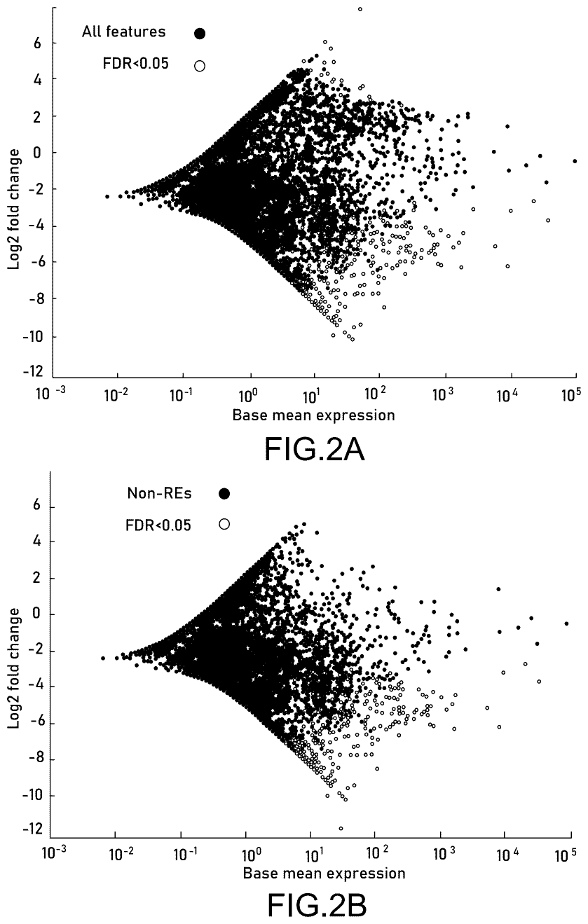

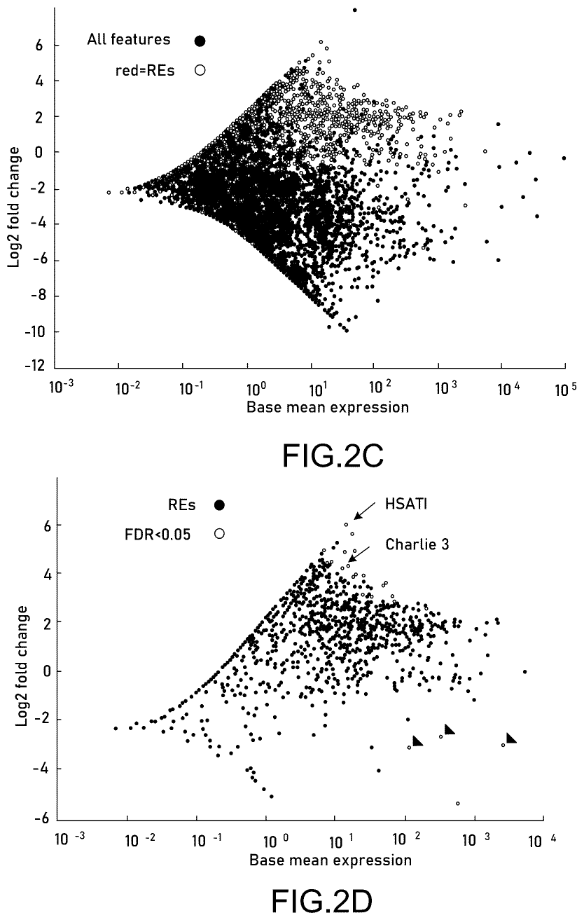

While these analyses revealed consistent over-representation of repetitive element sequences in OS serum EV preparations, the identities of the most over-represented elements were uncertain since programs that are not specifically designed for repetitive element detection may erroneously map repetitive element reads (33). To more accurately define the differential repetitive element representation, sequences were evaluated with TEtranscripts, which maps repetitive element sequences more accurately and quantitatively than non-dedicated programs (34). Using default settings with reads mapped to the GRCh38 genome, TEtranscripts confirmed that OS samples had far more significantly under-represented than over-represented sequences in comparison to control samples (FIG. 2A) and showed that most of the under-represented sequences were single copy genes (FIG. 2B). In contrast, a vast proportion of repetitive element sequences were over-represented in OS EV preparations (FIG. 2C) albeit with only 19 significantly overrepresented versus four significantly under-represented (FIG. 2D). Among significantly over-represented repeat elements, Human Satellite I (HSATI) had the highest fold change (log₂(6.14), p.adj=0.007) (FIG. 2D and Table 2, Analysis 1).

TABLE 2
Repeat Elements Significantly Differentially Represented in OS vs. Control EV Preparations
(p. adj <. 0.05; elements examined in the validation cohort underlined).

| Analysis 1 (mapped to GRChj38) | log₂FC | p. adj | Analysis 2 (mapped to hg19) | log₂FC | p.adj |
|---|---|---|---|---|---|
| HSATI:Satellite:Satellite | 6.14 | 0.007 | HSATII:Satellite:Satellite | 2.73 | 0.002 |
| LTR85a:Gypsy:LTR | 5.77 | 0.003 | LIMA6:L1:LINE | 2.65 | 0.041 |
| LTR75:ERVL:LTR | 5.07 | 0.042 | MSTC:ERVL-MaLR:LTR | 2.52 | 0.001 |
| LTR16E2:ERVL:LTR | 5.07 | 0.001 | (GAATG)n:Satellite:Satellite | 2.51 | 0.035 |
| LTR16A1:ERVL:LTR | 5.01 | 0.014 | Charlie4z:hAT-Charlie:DNA | 2.37 | 0.011 |
| Tigger8:TcMar-Tigger:DNA | 4.60 | 0.025 | Harlequin-int:ERV1:LTR | 2.28 | 0.032 |
| MLT1H2-int:ER VL-MaLR:LTR | 4.54 | 0.016 | L1M3:L1:LINE | 2.05 | 0.038 |
| BLACKJACK:hAT-Blackjack:DNA | 4.43 | 0.029 | L1MB8:L1:LINE | 2.03 | 0.005 |
| Charlie3:hAT-Charlie:DNA | 4.36 | 0.030 | L1PB4:L1:LINE | 2.00 | 0.021 |
| MER113:hAT-Charlie:DNA | 4.11 | 0.019 | L1MC3:L1:LINE | 1.90 | 0.046 |
| MLT1A1-int:ER VL-MaLR:LTR | 4.05 | 0.015 | L1PB2:L1:LINE | 1.89 | 0.026 |
| L1MCb:L1:LINE | 3.98 | 0.037 | L1MEc:L1:LINE | 1.88 | 0.047 |
| MLT2B2:ERVL:LTR | 3.75 | 0.018 | MLT1A0:ERVL-MaLR:LTR | 1.81 | 0.016 |
| HSAT4:centr:Satellite | 3.63 | 0.025 | L1MB7:L1:LINE | 1.80 | 0.028 |
| LTR32:ERVL:LTR | 3.25 | 0.042 | L1MDa:L1:LINE | 1.45 | 0.041 |
| Charlie4z:hAT-Charlie:DNA | 3.20 | 0.034 | L1PA12:L1:LINE | −2.27 | 0.002 |
| CER:Satellite:Satellite | 3.18 | 0.021 | | | |
| MLT1E2:ERVL-MaLR:LTR | 2.92 | 0.029 | | | |
| L1MC4a:L1:LINE | 2.71 | 0.040 | | | |
| MER74A:ERVL:LTR | −2.61 | 0.041 | | | |
| L1PA16:L1:LINE | −2.96 | 0.029 | | | |
| FAM:Alu:SINE | −3.05 | 0.015 | | | |
| FordPrefect:hAT-Tip100:DNA | −5.34 | <0.001 | | | |

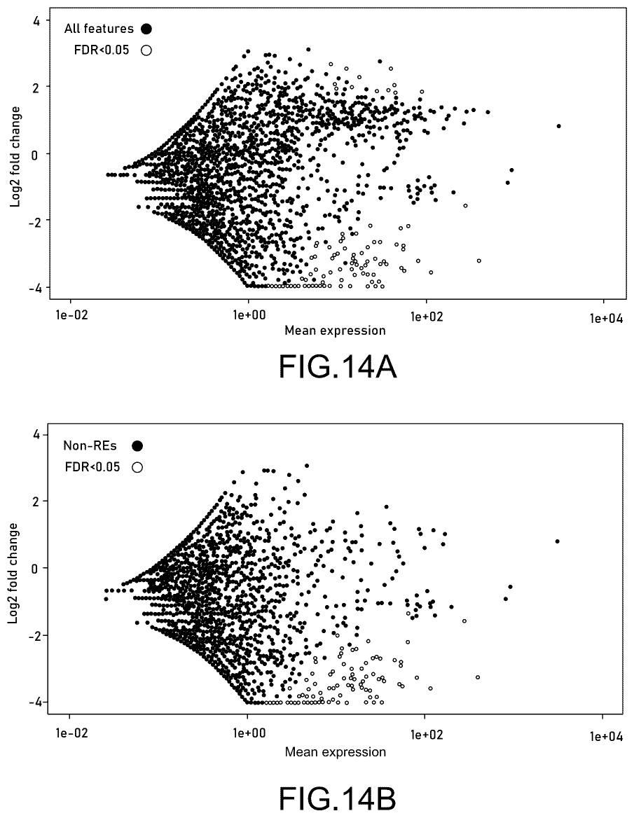

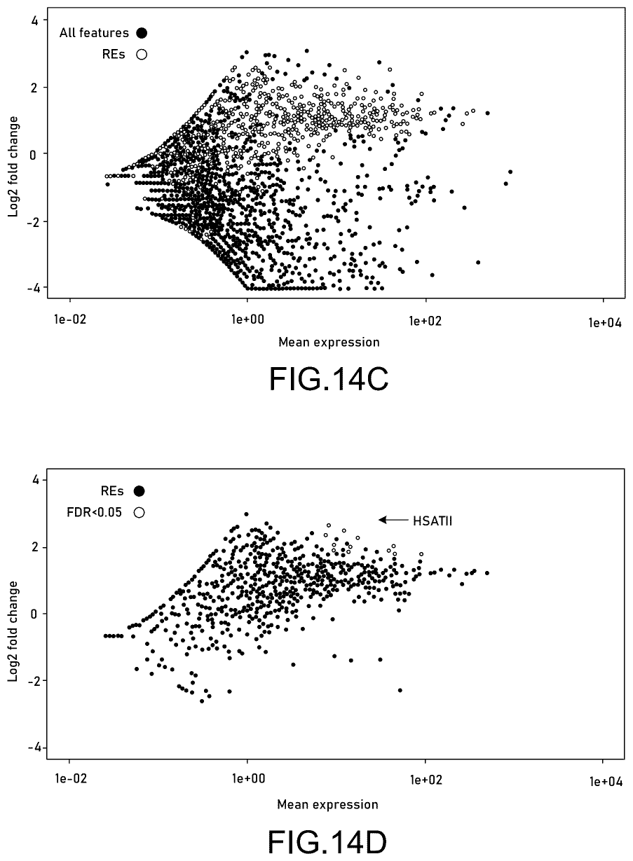

Because GRCh38 contains numerous alternative assemblies that are enriched for repetitive elements that might siphon repetitive element reads, adds synthetic centromeric repeat sequences, and hard-masks certain centromeric and genomic repeat arrays (35), it was considered whether these features might affect the ability to detect differential representation of unique or repetitive element sequences. To address this possibility, TEtranscripts analysis was re-performed with reads aligned to hg19, which lacks the GRCh38 alternative assemblies. This identified 15 significantly over-represented repeat elements, of which Human Satellite II (HSATII) had highest fold change (log₂(2.73), p.adj=0.002), and one significantly under-represented element in OS versus control sequences (Table 2, Analysis 2 and FIG. 14). The significantly differentially represented repetitive elements identified using hg19 had little overlap with those identified when mapping to GRCh38.

To illustrate the significant over-representation of RE DNA sequences as a proportion of all sequences in the above analysis we plotted the proportional expression of each sample expressed as read counts per million (CPM) of HSATII and LINE1 family sequences (FIG. 7, panel a, b). It was observed that the proportions of HSATII and LINE1 family reads relative to total reads (CPM) were increased with ROC curve AUC values of 1.0 and 0.986, respectively. To illustrate the significant over-representation of the average of the base-means of the 15 over-represented RE DNAs identified via hg19 mapping (Table 2, Analysis 2), we plotted the average of these base means for each sample, which yielded a ROC curve AUC value of 0.99 (FIG. 7, panel c).

In addition to identifying 15 over-represented RE DNAs the TEtranscript analysis identified 138 under-represented DNAs (Table 2, Analysis 2 and FIG. 14). To illustrate the significant differences in the ratio of the average base means of the 15 over-represented RE DNAs vs the 138 under-represented DNAs we plotted the ratio of these base mean averages for each sample (FIG. 7, panel d), which yielded a ROC curve AUC value of 1.0.

### Validation of Over-Representation of Repeat Elements in OS EV Preparations

It was next examined whether the increased representation of repetitive elements was evident in a validation set of mostly distinct samples. The validation cohort consisted of treatment-naïve OS patients from CHLA and HLOH including males and females between 7 and 46 years old and presenting with various OS types as well as approximately age-matched healthy individuals (Table 1). The validation cohort was independent of the discovery cohort except for re-analysis of OS1 and OS3, which were the only samples with a sufficient quantity to re-test. To assess the repetitive element over-representation, EV-associated nucleic acids were isolated and evaluated using methods that differed from the discovery cohort analyses: EVs were isolated by PEG6000 precipitation (36) instead of ExoQuick, nucleic acids were extracted using the miRNeasy micro-RNA extraction kit (Qiagen) instead of SeraMir, and repetitive elements were examined by reverse transcription and quantitative PCR (RT-qPCR) instead of sequencing. Similar to the discovery cohort, EV concentrations were not significantly higher in sera from OS patients compared to controls (data not shown).

RT-qPCR was used to analyze four representative repetitive element categories including the HSATI and HSATII satellite sequences that were most differentially overrepresented in TEtranscripts Analyses 1 and 2 (Table 2), the LINE1 P1 family member (L1P1) that showed the highest fold change in the RepeatMasker analysis (FIG. 13), and Charlie 3, another over-represented repetitive element with a significant log₂ fold change of 4.36 (20.5-fold increase) in TEtranscripts Analysis 1 (Table 2, FIG. 2D). RT-qPCR reactions yielded the predicted product sizes for HSATI (406 bp), L1P1 (83 bp), and Charlie 3 (104 bp). RT-qPCR of HSATII yielded prominent products of 85, 134, 183 and 281 bp, in agreement with HSATII genomic structure (FIG. 15), instead of a reported ˜200 bp amplicon found by RT-PCR with the same primers in an OS cell line (37). The HSATII and Charlie 3 products were confirmed to represent the predicted sequences by Sanger sequencing.

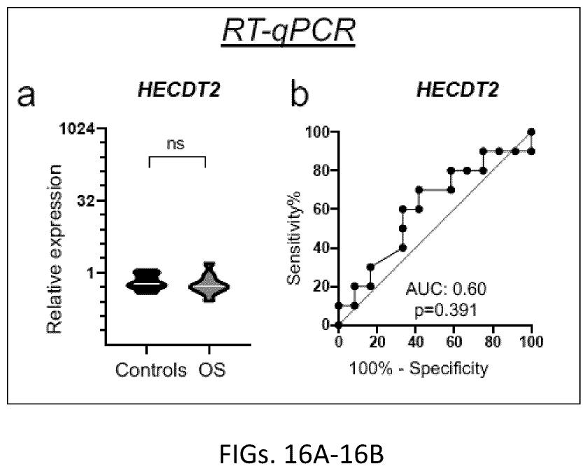

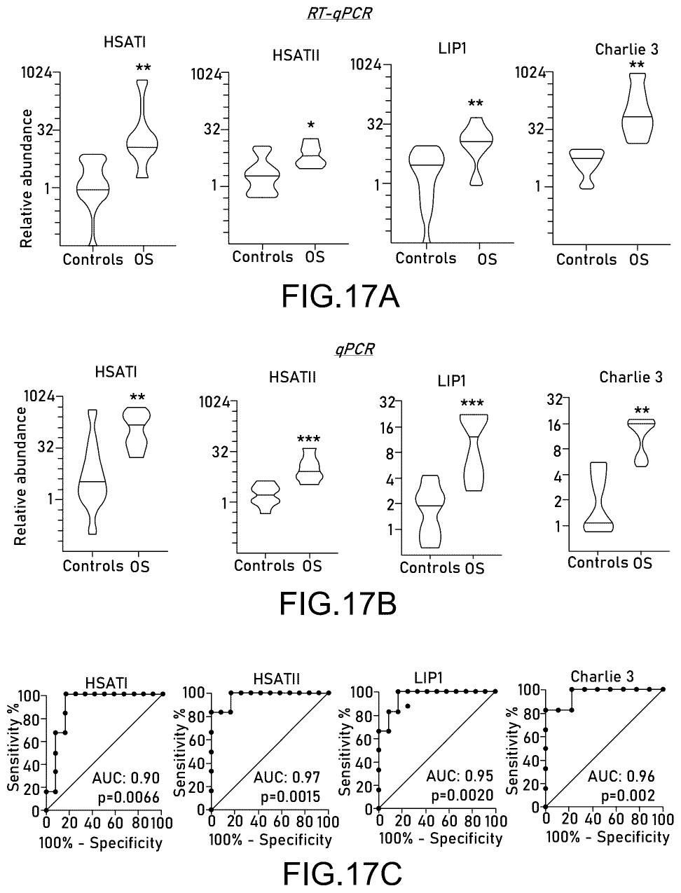

For each sample, RT-qPCR was performed on the same proportion of total EV nucleic acid extracted from the same serum volume and was normalized against a spike in RNA. The analyses confirmed the over-representation of HSATI, HSATII, L1P1, and Charlie 3 sequences in OS EV preparations (12.42-fold, p=0.0040; 3.33-fold, p=0.062; 3.56-fold, p=0.016; 12.6-fold, p=0.0007; respectively) (FIG. 3A). In contrast, the single copy gene HECDT2, chosen on the basis of a 4.67 log₂ fold change in TEtranscripts analysis, did not show a significant difference (FIGS. 16A and B), and was deemed to have been spuriously identified, possibly due to biases in the library construction method (38). The over-representation of HSATI, HSATII, L1P1, and Charlie 3 sequences was similar after removal of the OS1 and 053 samples that were also used in the discovery cohort (FIG. 17A). Thus, the over-representation of repetitive element sequences initially detected by nucleic acid sequencing was confirmed in a validation cohort using RT-qPCR.

### Over-Representation of Repetitive Element DNA but not RNA in OS EV Preparations

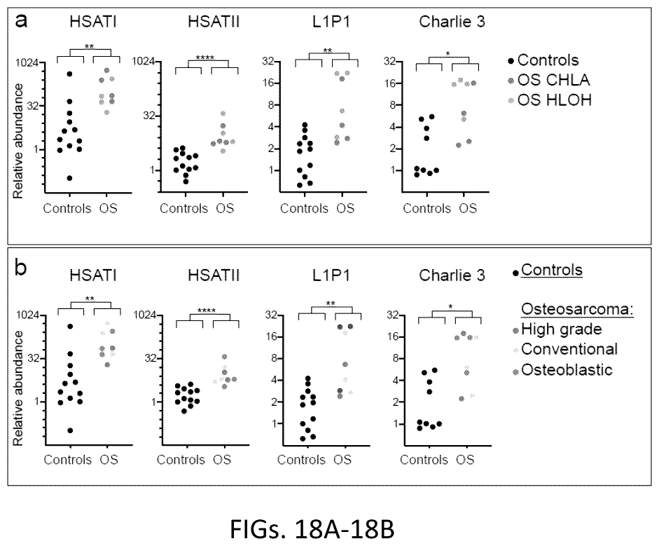

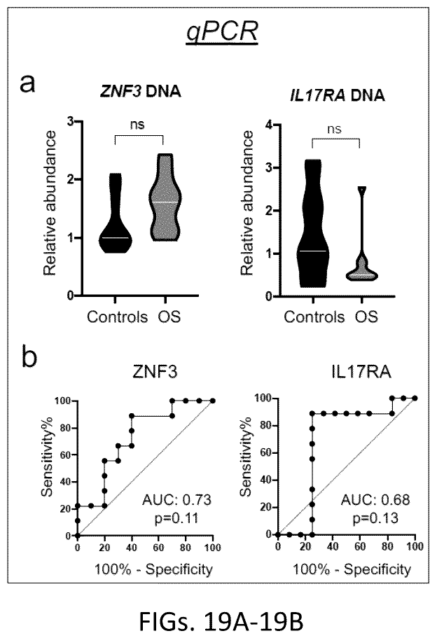

As the instant nucleic acid isolation and analysis methods could detect RNA as well as DNA sequences, the nucleic acid origin of the over-represented repetitive element sequences was examined by performing qPCR without reverse transcription. With this approach, the HSATI, HSATII, L1P1 and Charlie 3 amplification signals were significantly overrepresented in OS versus control EVs (22.18-fold, p=0.0015; 3.7-fold, p<0.0001; 2.86-fold, p=0.0015; 10.29-fold, p=0.011; respectively) (FIG. 3B), indicative of differential representation of repetitive element DNA, rather than RNA. Evaluation of the sensitivity and specificity by Receiver Operating Characteristic (ROC) curves yielded area under the curves (AUCs) of 0.86 or greater for each repetitive element (FIG. 3C). As for the RTqPCR analyses, results were similar after removal of OS1 and 053 (FIGS. 17B and C). HSATI, HSATII, L1P1 and Charlie 3 DNAs were similarly increased in OS samples from USA (CHLA) and China (HLOH) and in high-grade as well as non-high-grade OS samples (FIG. 18). In contrast, qPCR of/L/7RA and ZNF3 sequences, which are within 1 Mb of HSATI on chromosome 22 (chr22q11.21) and within 40 Mb of HSATII on chromosome 7 (chr7q22.1), respectively, did not show a significant difference between OS and control EVs (FIGS. 19A and B). Thus, major repetitive elements DNAs (HSATI, HSATII, L1P1, Charlie 3) were overrepresented, whereas single copy genes were not over-represented, in OS EV preparations.

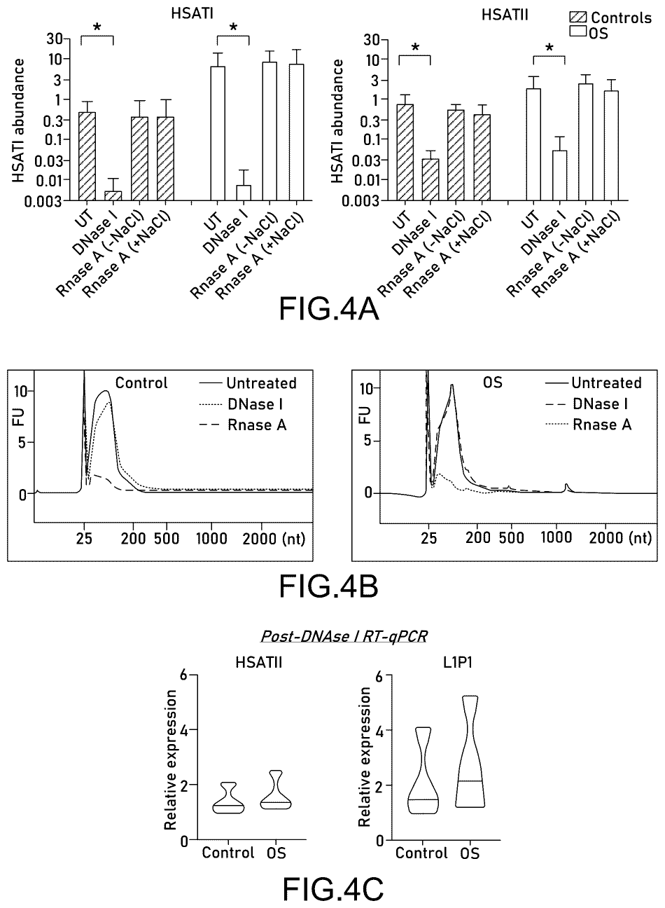

To further evaluate the abundance of repetitive element DNAs and control for possible artefactual generation of RT-independent products, PEG-precipitated EV preparations from four OS and four control sera were treated with DNase I or RNase A prior to nucleic acid extraction. RNase A treatments were performed in 1 M NaCl in order to cleave single-stranded RNA as well as in the absence of NaCl in order to cleave single-stranded and double-stranded RNA and RNA strands in RNA-DNA hybrids (39). After these treatments, nucleic acids were extracted with the miRNeasy Micro kit and HSATI and HSATII abundance were assessed by qPCR. In these analyses, DNase I treatment eliminated 97-99% of HSATI and 80-99% of HSATII signals in both OS and control samples, whereas RNase A treatments had no significant effect (FIG. 4A). Bioanalyzer assessments revealed that DNase treatment slightly decreased the amount of nucleic acid whereas RNase A eliminated most but not all of the nucleic acids (FIG. 4B), with the majority of the remaining nucleic acid likely representing protected EV RNA. The repetitive elements' sensitivity to DNase I prior to nucleic acid extraction implied that the repetitive element DNA sequences were not sequestered inside of EVs.

To assess whether OS serum EV preparations might also have an increased abundance of repetitive element RNAs, PEG-precipitated EVs were prepared, treated with DNase I, and the remaining nucleic acids extracted and examined by RT-qPCR. In these samples, no amplification signal was detected for HSATI or Charlie 3, while HSATII and L1P1 products were reduced ˜32-64-fold compared to non-DNase I treated samples and showed no significant difference in control and OS samples (FIG. 4C and data not shown). Thus, HSATI, HSATII, L1P1, and Charlie 3 DNAs were over-abundant in OS compared to control EV preparations whereas their RNAs were either undetectable or present in similar quantities.

Co-Purification of OS-Associated Repetitive Element DNAs with EVs in Size Exclusion Chromatography but not Exosome Immunoaffinity Capture

To further evaluate if repetitive element DNAs that were more abundant in OS patient PEG-precipitations (here termed ‘OS-associated’ repetitive element DNAs) are associated with EVs, it was examined whether they co-purified with EVs prepared by size exclusion chromatography (SEC) and exosome immunoaffinity capture. SEC yields purer EV populations (40,41) with lower protein contamination compared to PEG precipitation (42,43), whereas exosome immunoaffinity capture uses well-characterized surface markers CD9 or CD81 to highly purify intact exosomes (41,44).

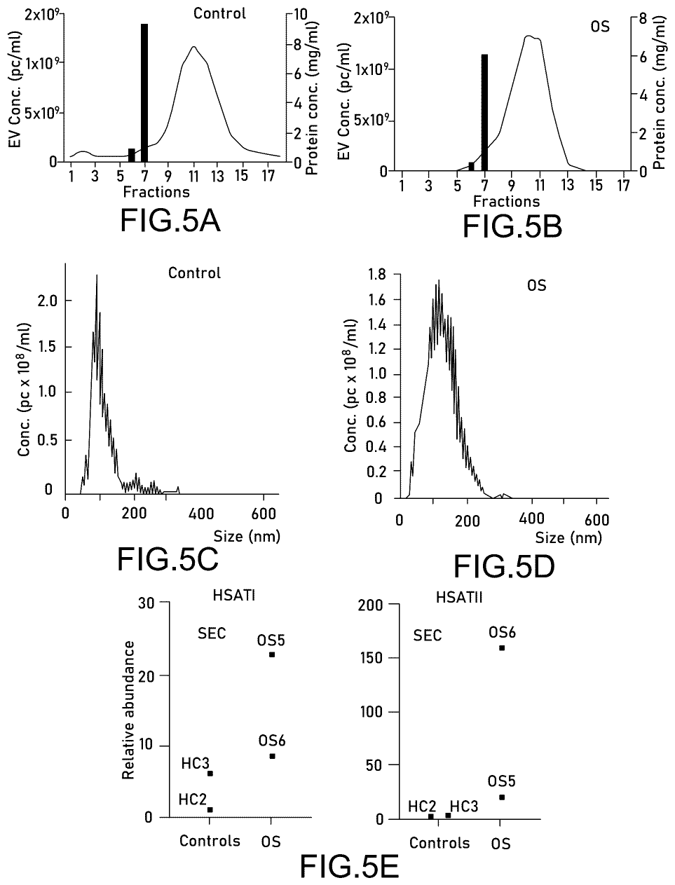

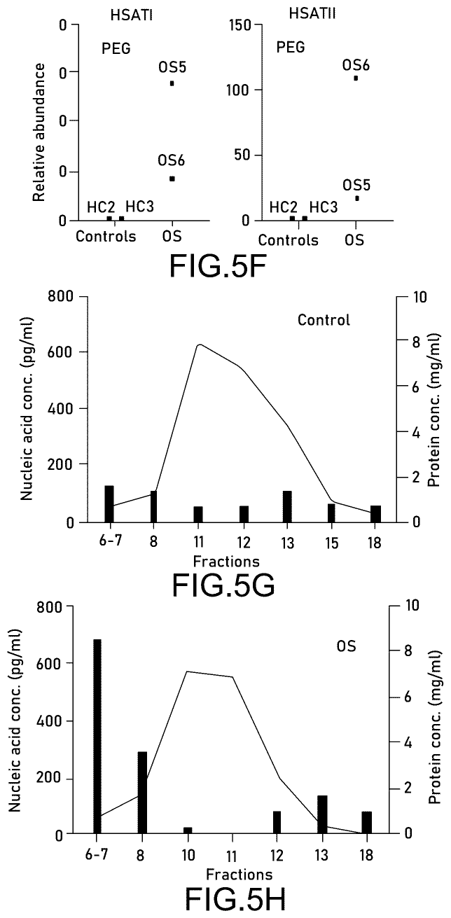

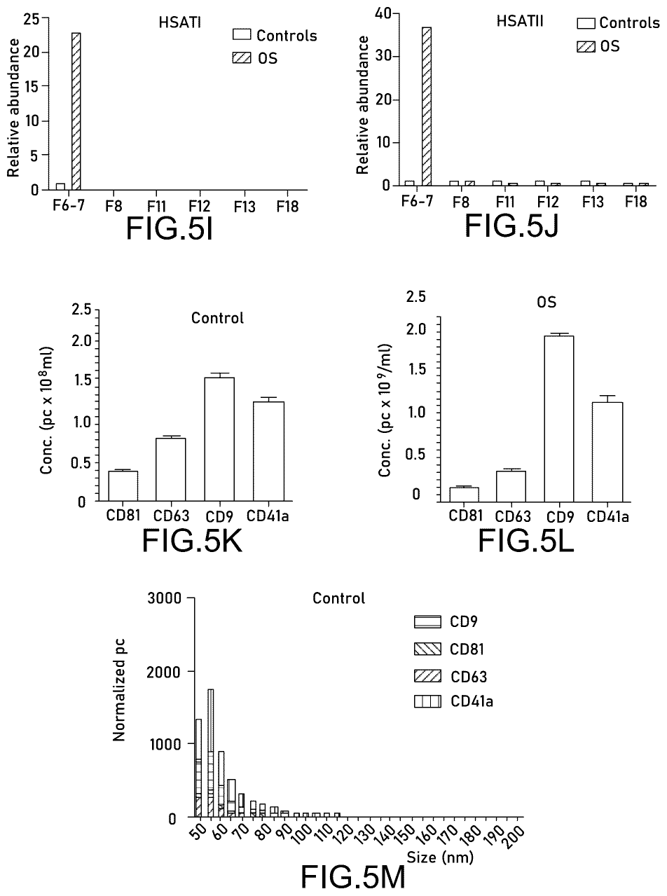

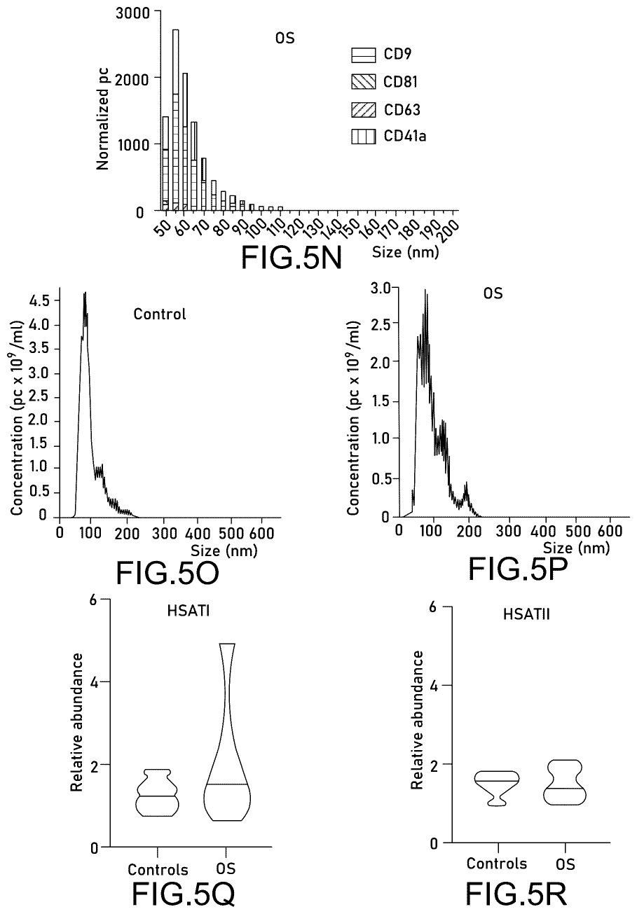

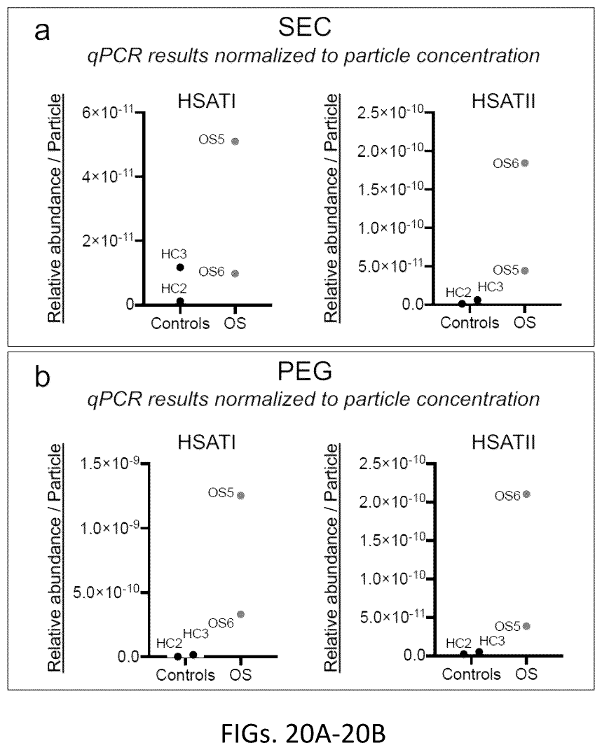

It was first examined if repetitive element DNAs co-purify with SEC-isolated EVs from two control and two OS samples. Nanoparticle tracking analyses revealed that the control and OS EVs both eluted from size exclusion columns solely in fractions 6 and 7 (FIGS. 5A and B) and had similar size profiles (FIGS. 5C and D). qPCR analyses revealed that the OS EV fractions had more abundant HSATI and HSATII DNA (FIG. 5E), as observed with the PEG-isolated EVs of the same samples (FIG. 5F). Similarly, HSATI and HSATII levels were higher in the same OS versus control sera when normalized to EV concentration (FIGS. 20A and B). Furthermore, nucleic acids obtained from arbitrarily selected non-EV fractions from one control and one OS fractionation (FIGS. 5G and H) revealed no detectable HSATI and only minimal HSATII, which was not higher in OS samples (FIGS. 5I and J). Thus, HSATI and HSATII DNAs co-purified with EVs in SEC with a greater abundance in OS compared to control sera, similar to that of PEG preparations.

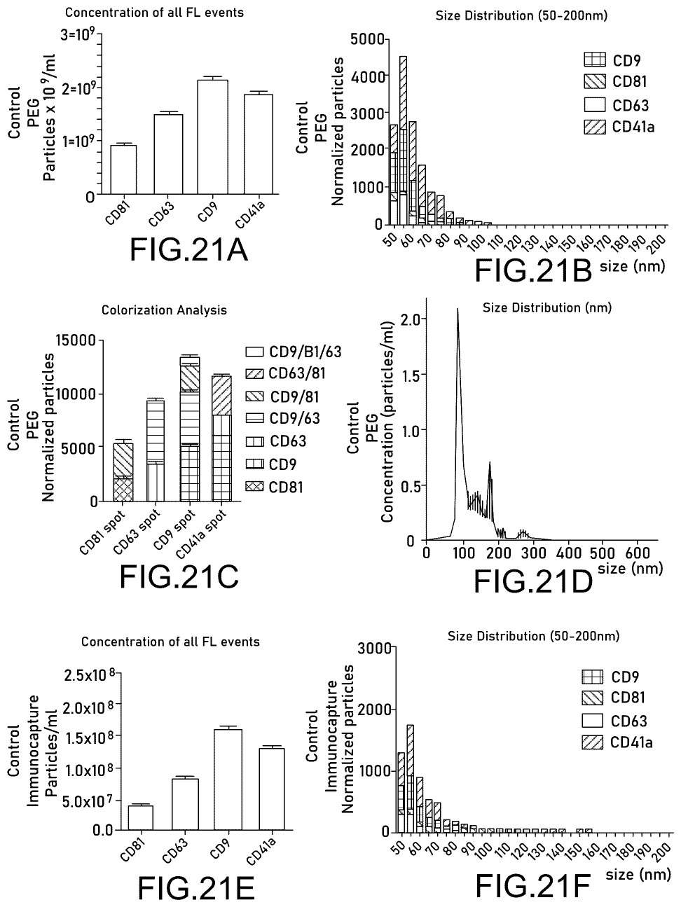

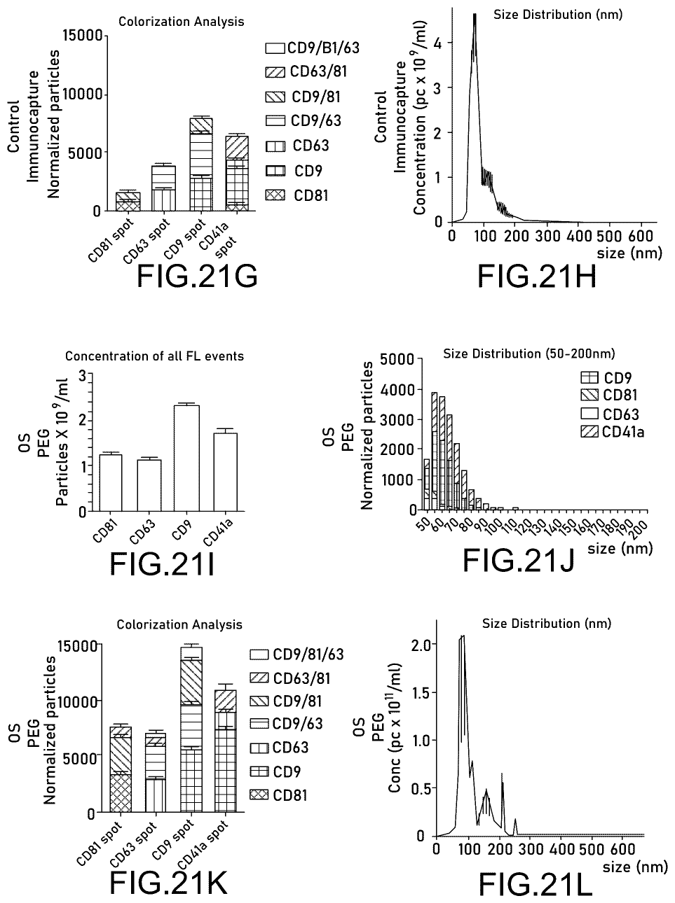

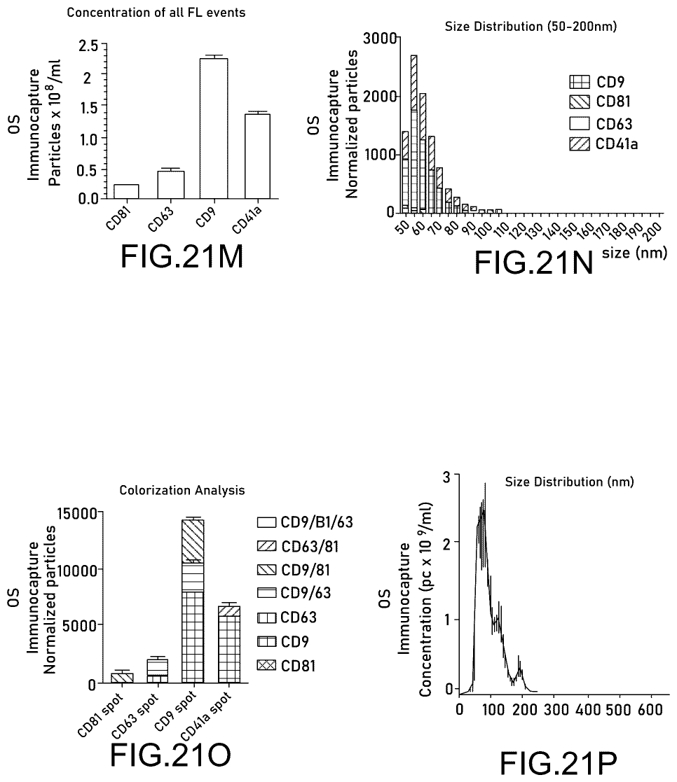

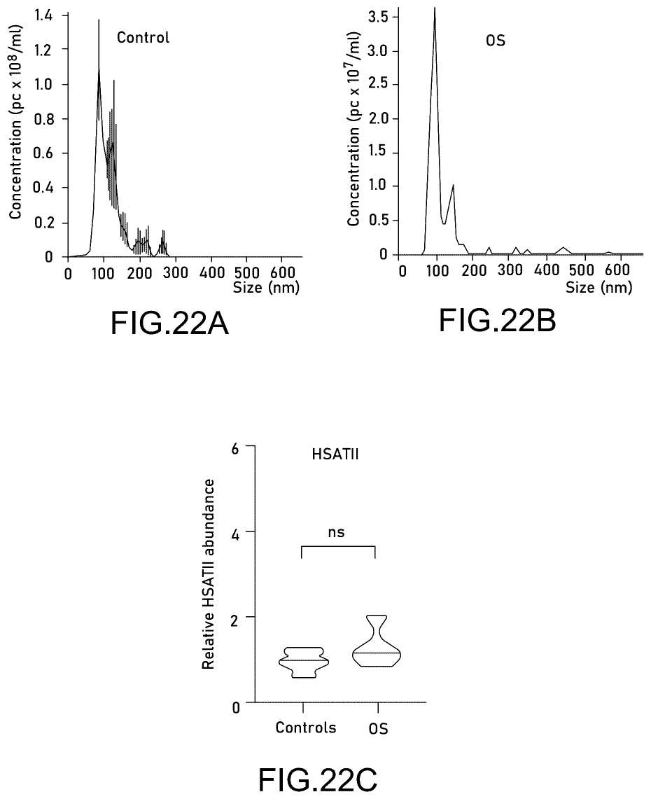

It was next assessed whether OS-associated repetitive element DNAs co-purified with EVs in exosome CD9 or CD81 immunoaffinity capture. In pilot studies, it was confirmed that the immunocapture approach enriched for exosomes by single particle interferometric reflectance imaging sensing (SP-IRIS) using an ExoView instrument (45). SP-IRIS analyses showed that a similar number of EV particles eluted from CD9 immunoaffinity capture from control (21,006 particles, n=1) and OS sera (21,264±374 (SEM) particles, n=2), that the eluted particles could be re-immunocaptured on the microarray-based solid phase chip coated with antibodies to exosomal surface markers (FIGS. 5K and L), and that the re-captured particles were from 50 to 80 nm diameter (characteristic of exosomes) (FIGS. 5M and N) and expressed various combinations of exosomal markers CD81, CD63, and CD9, similar to the PEG precipitated EV preparations of the same samples (FIG. 21). Nanoparticle tracking analyses of the CD9 and CD81 immunoaffinity capture eluates showed particle size distributions similar to that of PEG precipitations but larger than reported by SP-IRIS (FIGS. 50 and P, FIGS. 22A and B), as expected (46). However, in contrast to PEG- and SEC-isolated EVs, CD9 and CD81 immunoaffinity captured EV preparations showed no significant difference in HSATI and HSATII DNA abundance between control and OS samples (FIGS. 5Q and R and FIG. 22C). Thus, the OS-associated HSATI and HSATII DNAs failed to co-purify with EVs during immunocapture in contrast to their co-purification with EVs isolated via PEG precipitation or SEC. It is inferred that OS-associated HSATI and HSATII DNAs either fail to bind CD9+ or CD81+ exosomes or dissociate from such exosomes under immunocapture conditions. Enrichment of human satellite sequences in EV-associated DNA but not in total cfDNA in OS patient sera

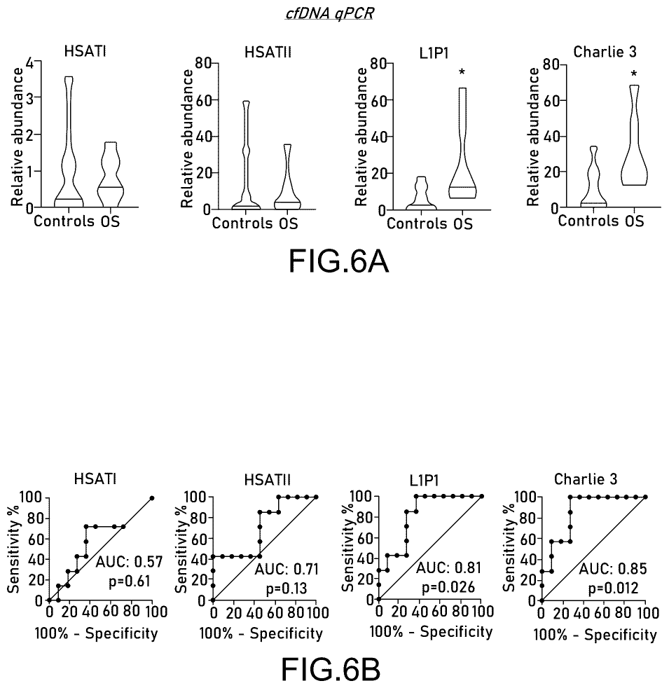

The finding that repetitive element DNAs were increased in OS patient PEG and SEC EV preparations yet not tightly bound to CD9+ or CD81+ exosomes raised the possibility that repetitive element DNAs might be more abundant in total cfDNA of OS patients and were proportionately present as contaminants in OS and control EV nucleic acid preparations. To examine this possibility, nucleic acids were extracted directly from equal volumes of OS and control sera using the same miRNeasy micro-RNA extraction kit as used for EV preparations and the repetitive element abundance was examined by qPCR. This revealed that L1P1 and Charlie 3 were significantly more abundant whereas HSATI and HSATII were present at similar levels in OS and control cfDNA samples (FIG. 6A). Omitting the PEG EV preparation step eliminated the diagnostic sensitivity of HSATI and HSATII (AUC=<0.72) while reducing that of L1P1 (AUC=0.81) and not affecting that of Charlie 3 (AUC=0.85) (FIG. 6B) as compared to the AUC values obtained after PEG precipitation (FIG. 3C). These data imply that EV enrichment by PEG or SEC was required in order to detect the increased representation of HSATI and HSATII and to increase the sensitivity for L1P1 in OS patient versus control sera.

The methods were furthered verified in breast cancer (FIG. 11). Four different RE DNAs were examined in cancer patient sera. FIG. 11 shows breast cancer results. The two breast cancer samples had increased HSATII (‘Sat2’), L1P1, and Charlie 3.

### DISCUSSION

Sensitive biomarkers are needed to detect incipient OS tumors and enable lifesaving interventions in predisposed individuals. Prior studies identified a variety of potential OS biomarkers yet none had sufficient sensitivity to enable reliable OS detection (12,13). To identify new OS biomarkers, nucleic acid sequences associated with circulating EVs in OS patients was investigated. This revealed an over-representation of diverse repetitive element sequences, among which human satellites HSATI and HSATII were the most significantly increased upon mapping to the GRCh38 and hg19 genome builds. The over-represented repetitive element sequences were confirmed in a validation cohort and found to reflect repetitive element DNAs that co-purified with circulating EVs but were not tightly bound to CD9+ or CD81+ exosomes. HSATI and HSATII were distinguished from other repetitive elements in that they were enriched in serum EV preparations but not in total cfDNA, implying that they segregated into distinct complexes in the circulation of OS patients.

The detection of increased repetitive element DNAs in OS patient sera was enabled by a novel screening approach. First, in the discovery cohort, serum EVs were prepared using a precipitation method that concentrates exosomes as well as other EVs and non-vesicular constituents (29,30), which enlarged the population of biomarker candidates. Second, EVs or nucleic acid preparations were not treated with DNase, which enabled isolation of DNAs as well as RNAs and further diversified the potential biomarker pool. Third, nucleic acids were isolated using small RNA preparation kits that also captured repetitive element DNAs, and then a sequencing library was built by direct ligation of adapters to extracted DNAs as well as RNAs, using an activity with properties similar to T4 RNA ligase (47), which allowed the discovery of differentially represented DNA as well as RNA species. Finally, repetitive element sequences were evaluated that include diverse satellite and non-satellite categories that may comprise more than two-thirds of the human genome (48). Repetitive elements are often ignored in human sequencing studies because of the complexity involved in properly aligning short sequencing reads to highly repetitive regions as well as poor understanding of their functional relevance (33). However, the paucity of over-represented single copy genes in OS patient EV preparations prompted consideration of whether repetitive element sequences might be over-represented.

To examine differential repetitive element sequence representation, RepeatMasker was initially used to align sequence reads against the Repbase library of known repeats (32). This revealed an over-representation of all repetitive element categories in OS serum EV preparations, with the LINE1 family member LIP1 as the most significantly overrepresented species (FIG. 13). To more accurately identify differentially represented repetitive elements, TEtranscripts was used, which assigns both uniquely and ambiguously mapped reads to all possible gene and transposable element-derived transcripts in order to statistically infer the correct gene or transposable element abundances (34). TEtranscripts analyses confirmed that repetitive elements were overrepresented in OS serum EV preparations and identified HSATI, HSATII and Charlie 3, among others, as significantly over-represented (Table 2). Different elements were identified when reads were aligned to GRch38 or to hg19, likely due to the presence of alternative repetitive-element-enriched sequence assemblies in GRch38 (35).

The increased abundance of selected repetitive element sequences was validated in a second patient cohort. In the validation set, their increased abundance was observed via qPCR, without reverse-transcription and in a DNase-sensitive manner, implying differential representation of repetitive element DNAs. Repetitive element DNA sequences were similarly increased in OS samples from USA (CHLA) and China (HLOH) and did not correlate with the OS grade, suggesting that these elements are produced independently of OS type. EV-associated repetitive element DNAs showed a high sensitivity and specificity for sera of patients with an OS diagnosis, with a significant AUC >0.9 for HSATI, HSATII and L1P1. However, the sensitivity was diminished by omitting the EV preparation step, particularly for HSATI and HSATII (FIG. 6), suggesting that these OS-associated repetitive element DNAs are segregated from bulk cfDNA.

The results show that EV-associated repetitive element DNAs are among the most sensitive markers of OS identified to date, with ROC curve AUCs of 0.90 for HSATI and 0.97 for HSATII (FIG. 3C). By comparison, in prior OS biomarker analyses, circulating miRNAs had at best ROC curve AUCs of 0.833-0.955, yet were significantly elevated in only a subset of many similar miRNA screens (11, 54-56). Likewise, a deep sequencing approach detected circulating tumor DNA aneuploidy in only 50% of treatment-naïve OS patients (12). Still, in this study, the increased repetitive element DNAs were observed in sera of already-diagnosed OS patients.

A final question raised by our findings is whether similar EV-associated repetitive element DNAs are increased in the circulation of patients with other cancers. Notably, centromeric and pericentric repetitive element RNA sequences, particularly alpha satellites and satellite II and III sequences, were reported to be overexpressed in testicular, liver, ovarian, and lung cancers compared to corresponding normal tissues (57). The pericentric human satellite II (HSATII) RNA was reported to be the most differentially expressed satellite subfamily in pancreatic cancer tissue and was also overexpressed in lung, kidney, ovarian, colon and prostate cancers (58, 59). Moreover, HSATII was one of the six most up-regulated satellite sequences in a study comparing fresh bone and OS samples by RNA-seq (28). LINE-1 was also overexpressed in pancreatic and prostate tumor samples (60, 61). However, although LINE1 and other repetitive element RNAs were detected in cancer cell-derived EVs in culture (27), their up-regulation has not been reported for circulating cell-free RNA in cancer patients. Thus, circulating repetitive element DNAs are enriched in additional cancer types and can be quantified/detected by the assays/methods described herein.

### BIBLIOGRAPHY

1 Damron, T. A., Ward, W. G. & Stewart, A. Osteosarcoma, chondrosarcoma, and
   Ewing's sarcoma: National Cancer Data Base Report. Clin Orthop Relat Res 459, 40-47, doi:10.1097/BLO.0b013e318059b8c9 (2007).
2 Ottaviani, G. & Jaffe, N. The epidemiology of osteosarcoma. Cancer Treat Res 152, 3-13, doi:10.1007/978-1-4419-0284-9_1 (2009).
3 Dorfman, H. D. & Czerniak, B. Bone cancers. Cancer 75, 203-210, doi:10.1002/1097-0142(19950101)75:1+<203::aidcncr2820751308>3.0.co; 2-v (1995).
4 Misaghi, A., Goldin, A., Awad, M. & Kulidjian, A. A. Osteosarcoma: a comprehensive review. SICOT J 4, 12, doi:10.1051/sicotj/2017028 (2018).
5 Kleinerman, R. A. et al. Risk of new cancers after radiotherapy in long-term survivors of retinoblastoma: an extended follow-up. J Clin Oncol 23, 2272-2279, doi:10.1200/JCO.2005.05.054 (2005).
6 Zumarraga, J. P., Baptista, A. M., Rosa, L. P., Caiero, M. T. & Camargo, P. Serum Values of Alkaline Phosphatase and Lactate Dehydrogenase in Osteosarcoma. Acta Ortop Bras 24, 142-146, doi:10.1590/1413-785220162403157033 (2016).
7 Gu, J. et al. Identification of osteosarcoma-related specific proteins in serum samples using surface-enhanced laser desorption/ionization-time-of-flight mass spectrometry. J Immunol Res 2014, 649075, doi:10.1155/2014/649075 (2014).
8 Bottani, M., Banfi, G. & Lombardi, G. Circulating miRNAs as Diagnostic and Prognostic Biomarkers in Common Solid Tumors: Focus on Lung, Breast, Prostate Cancers, and Osteosarcoma. J Clin Med 8, doi:10.3390/jcm8101661 (2019).
9 Raimondi, L. et al. Circulating biomarkers in osteosarcoma: new translational tools for diagnosis and treatment. Oncotarget 8, 100831-100851, doi:10.18632/oncotarget.19852 (2017).
10 Viera, G. M. et al. miRNA signatures in childhood sarcomas and their clinical implications. Clin Transl Oncol 21, 1583-1623, doi:10.1007/s12094-019-02104-z (2019).
11 Nakka, M. et al. Biomarker significance of plasma and tumor miR-21, miR-221, and miR-106a in osteosarcoma. Oncotarget 8, 96738-96752, doi:10.18632/oncotarget.18236 (2017).
12 Klega, K. et al. Detection of Somatic Structural Variants Enables Quantification and Characterization of Circulating Tumor DNA in Children With Solid Tumors. JCO Precis Oncol 2018, doi:10.1200/PO.17.00285 (2018).
13 Zamborsky, R., Kokavec, M., Harsanyi, S. & Danisovic, L. Identification of Prognostic and Predictive Osteosarcoma Biomarkers. Med Sci (Basel) 7, doi:10.3390/medsci7020028 (2019).
14 De Rubis, G., Rajeev Krishnan, S. & Bebawy, M. Liquid Biopsies in Cancer Diagnosis, Monitoring, and Prognosis. Trends Pharmacol Sci 40, 172-186, doi:10.1016/j.tips.2019.01.006 (2019).
15 Kalluri, R. The biology and function of exosomes in cancer. J Clin Invest 126, 1208-1215, doi:10.1172/JC181135 (2016).
16 Logozzi, M. et al. High levels of exosomes expressing CD63 and caveolin-1 in plasma of melanoma patients. PLoS One 4, e5219, doi:10.1371/journal.pone.0005219 (2009).
17 O'Brien, K. et al. Exosomes from triple-negative breast cancer cells can transfer phenotypic traits representing their cells of origin to secondary cells. Eur J Cancer 49, 1845-1859, doi:10.1016/j.ejca.2013.01.017 (2013).
18 Kahlert, C. & Kalluri, R. Exosomes in tumor microenvironment influence cancer progression and metastasis. J Mol Med (Berl) 91, 431-437, doi:10.1007/s00109-013-1020-6 (2013).
19 Jalalian, S. H., Ramezani, M., Jalalian, S. A., Abnous, K. & Taghdisi, S. M. Exosomes, new biomarkers in early cancer detection. Anal Biochem 571, 1-13, doi:10.1016/j.ab.2019.02.013 (2019).
20 Lotvall, J. et al. Minimal experimental requirements for definition of extracellular vesicles and their functions: a position statement from the International Society for Extracellular Vesicles. J Extracell Vesicles 3, 26913, doi:10.3402/jev.v3.26913 (2014).
21 Cocucci, E. & Meldolesi, J. Ectosomes and exosomes: shedding the confusion between extracellular vesicles. Trends Cell Biol 25, 364-372, doi:10.1016/j.tcb.2015.01.004 (2015).
22 Ludwig, A. K. & Giebel, B. Exosomes: small vesicles participating in intercellular communication. Int J Biochem Cell Biol 44, 11-15, doi:10.1016/j.bioce1.2011.10.005 (2012).
23 Valadi, H. et al. Exosome-mediated transfer of mRNAs and microRNAs is a novel mechanism of genetic exchange between cells. Nat Cell Biol 9, 654-659, doi:10.1038/ncb1596 (2007).
24 Haraszti, R. A. et al. High-resolution proteomic and lipidomic analysis of exosomes and microvesicles from different cell sources. J Extracell Vesicles 5, 32570, doi:10.3402/jev.v5.32570 (2016).
25 Corrado, C. et al. Exosomes as intercellular signaling organelles involved in health and disease: basic science and clinical applications. Int J Mol Sci 14, 5338-5366, doi:10.3390/ijms14035338 (2013).
26 Whiteside, T. L. Tumor-Derived Exosomes and Their Role in Cancer Progression. Adv Clin Chem 74, 103-141, doi:10.1016/bs.acc.2015.12.005 (2016).
27 Balaj, L. et al. Tumour microvesicles contain retrotransposon elements and amplified oncogene sequences. Nat Commun 2, 180, doi:10.1038/ncomms1180 (2011).
28 Ho, X. D. et al. Analysis of the Expression of Repetitive DNA Elements in Osteosarcoma. Front Genet 8, 193, doi:10.3389/fgene.2017.00193 (2017).
29 Lobb, R. J. et al. Optimized exosome isolation protocol for cell culture supernatant and human plasma. J Extracell Vesicles 4, 27031, doi:10.3402/jev.v4.27031 (2015).
30 Tang, Y. T. et al. Comparison of isolation methods of exosomes and exosomal RNA from cell culture medium and serum. Int J Mol Med 40, 834-844, doi:10.3892/ijmm.2017.3080 (2017).
31 Love, M. I., Huber, W. & Anders, S. Moderated estimation of fold change and dispersion for RNA-seq data with DESeq2. Genome Biol 15, 550, doi:10.1186/s13059-014-0550-8 (2014).
32 Tarailo-Graovac, M. & Chen, N. Using RepeatMasker to identify repetitive elements in genomic sequences. Curr Protoc Bioinformatics Chapter 4, Unit 4 10, doi:10.1002/0471250953.bi0410s25 (2009).
33 Slotkin, R. K. The case for not masking away repetitive DNA. Mob DNA 9, 15, doi:10.1186/s13100-018-0120-9 (2018).
34 Jin, Y., Tam, 0. H., Paniagua, E. & Hammell, M. TEtranscripts: a package for including transposable elements in differential expression analysis of RNAseq datasets. Bioinformatics 31, 3593-3599, doi:10.1093/bioinformatics/btv422 (2015).
35 Church, D. M. et al. Extending reference assembly models. Genome Biol 16, 13, doi:10.1186/s13059-015-0587-3 (2015).
36 Rider, M. A., Hurwitz, S. N. & Meckes, D. G., Jr. ExtraPEG: A Polyethylene Glycol-Based Method for Enrichment of Extracellular Vesicles. Sci Rep 6, 23978, doi:10.1038/srep23978 (2016).
37 Hall, L. L. et al. Demethylated HSATII DNA and HSATII RNA Foci Sequester PRC1 and MeCP2 into Cancer-Specific Nuclear Bodies. Cell Rep 18, 2943-2956, doi:10.1016/j.celrep.2017.02.072 (2017).
38 Fuchs, R. T., Sun, Z., Zhuang, F. & Robb, G. B. Bias in ligation-based small RNA sequencing library construction is determined by adaptor and RNA structure. PLoS One 10, e0126049, doi:10.1371/journal.pone.0126049 (2015).
39 Ausubel, M., R. Brent, R. E. Kingston, D. D. Moore, J. G. Seidman, J. A. Smith, and K. Struhl. Current protocols in molecular biology. Volumes 1 and 2. John Wiley & Sons, Inc., Media, P A, 1988. Molecular Reproduction and Development 1, 146-146, doi:10.1002/mrd.1080010210.
40 Nordin, J. Z. et al. Ultrafiltration with size-exclusion liquid chromatography for high yield isolation of extracellular vesicles preserving intact biophysical and functional properties. Nanomedicine 11, 879-883, doi:10.1016/j.nano.2015.01.003 (2015).
41 Taylor, D. D. & Shah, S. Methods of isolating extracellular vesicles impact down-stream analyses of their cargoes. Methods 87, 3-10, doi:10.1016/j.ymeth.2015.02.019 (2015).
42 Andreu, Z. et al. Comparative analysis of EV isolation procedures for miRNAs detection in serum samples. J Extracell Vesicles 5, 31655, doi:10.3402/jev.v5.31655 (2016).
43 Konoshenko, M. Y., Lekchnov, E. A., Vlassov, A. V. & Laktionov, P. P. Isolation of Extracellular Vesicles: General Methodologies and Latest Trends. Biomed Res Int 2018, 8545347, doi:10.1155/2018/8545347 (2018).
44 Greening, D. W., Xu, R., Ji, H., Tauro, B. J. & Simpson, R. J. A protocol for exosome isolation and characterization: evaluation of ultracentrifugation, density-gradient separation, and immunoaffinity capture methods. Methods Mol Biol 1295, 179-209, doi:10.1007/978-1-4939-2550-6_15 (2015).
45 Daaboul, G. G. et al. Digital Detection of Exosomes by Interferometric Imaging. Sci Rep 6, 37246, doi:10.1038/srep37246 (2016).
46 Bachurski, D. et al. Extracellular vesicle measurements with nanoparticle tracking analysis—An accuracy and repeatability comparison between NanoSight NS300 and ZetaView. J Extracell Vesicles 8, 1596016, doi:10.1080/20013078.2019.1596016 (2019).
47 Higgins, N. P., Geballe, A. P. & Cozzarelli, N. R. Addition of oligonucleotides to the 5′-terminus of DNA by T4 RNA ligase. Nucleic Acids Res 6, 1013-1024, doi:10.1093/nar/6.3.1013 (1979).
48 de Koning, A. P., Gu, W., Castoe, T. A., Batzer, M. A. & Pollock, D. D. Repetitive elements may comprise over two-thirds of the human genome. PLoS Genet 7, e1002384, doi:10.1371/journal.pgen.1002384 (2011).
49 Bronkhorst, A. J. et al. Sequence analysis of cell-free DNA derived from cultured human bone osteosarcoma (143B) cells. Tumour Biol 40, 1010428318801190, doi:10.1177/1010428318801190 (2018).
50 Montermini, L. et al. Inhibition of oncogenic epidermal growth factor receptor kinase triggers release of exosome-like extracellular vesicles and impacts their phosphoprotein and DNA content. J Biol Chem 290, 24534-24546, doi:10.1074/jbc.M115.679217 (2015).
51 Zhang, H. et al. Identification of distinct nanoparticles and subsets of extracellular vesicles by asymmetric flow field-flow fractionation. Nat Cell Biol 332-343, doi:10.1038/s41556-018-0040-4 (2018).
52 Takahashi, A. et al. Exosomes maintain cellular homeostasis by excreting harmful DNA from cells. Nat Commun 8, 15287, doi:10.1038/ncomms15287 (2017).
53 Jeppesen, D. K. et al. Reassessment of Exosome Composition. Cell 177, 428-445 e418, doi:10.1016/j.cell.2019.02.029 (2019).
54 Ouyang, L. et al. A three-plasma miRNA signature serves as novel biomarkers for osteosarcoma. Med Oncol 30, 340, doi:10.1007/s12032-012-0340-7 (2013).
55 Yuan, J., Chen, L., Chen, X., Sun, W. & Zhou, X. Identification of serum microRNA-21 as a biomarker for chemosensitivity and prognosis in human osteosarcoma. J Int Med Res 40, 2090-2097, doi:10.1177/030006051204000606 (2012).
56 Yang, Z. et al. Serum microRNA-221 functions as a potential diagnostic and prognostic marker for patients with osteosarcoma. Biomed Pharmacother 75, 153-158, doi:10.1016/j.biopha.2015.07.018 (2015).
57 Eymery, A. et al. A transcriptomic analysis of human centromeric and pericentric sequences in normal and tumor cells. Nucleic Acids Res 37, 6340-6354, doi:10.1093/nar/gkp639 (2009).
58 Ting, D. T. et al. Aberrant overexpression of satellite repeats in pancreatic and other epithelial cancers. Science 331, 593-596, doi:10.1126/science.1200801 (2011).
59 Bersani, F. et al. Pericentromeric satellite repeat expansions through RNA derived DNA intermediates in cancer. Proc Natl Acad Sci USA 112, 15148-15153, doi:10.1073/pnas.1518008112 (2015).
60 Contreras-Galindo, R. et al. Human endogenous retrovirus K (HML-2) elements in the plasma of people with lymphoma and breast cancer. J Virol 82, 9329-9336, doi:10.1128/JVI.00646-08 (2008).
61 Criscione, S. W., Zhang, Y., Thompson, W., Sedivy, J. M. & Neretti, N. Transcriptional landscape of repetitive elements in normal and cancer human cells. BMC Genomics 15, 583, doi:10.1186/1471-2164-15-583 (2014).
62 Babcock, M., Yatsenko, S., Stankiewicz, P., Lupski, J. R. & Morrow, B. E. AT-rich repeats associated with chromosome 22q11.2 rearrangement disorders shape human genome architecture on Yq12. Genome Res 17, 451-460, doi:10.1101/gr.5651507 (2007).

The invention is described with reference to various specific and preferred embodiments and techniques. However, it should be understood that many variations and modifications may be made while remaining within its scope. All referenced publications, patents and patent documents are intended to be incorporated by reference, as though individually incorporated by reference.

1. A method to selectively enrich for associated repetitive element (RE) DNAs in a sample comprising:
a) obtaining a blood or serum sample from a subject; and
b) enriching the sample of a) for extracellular vesicles (EVs), and
c) isolating small nucleic acids from b) so as to enrich for associated RE DNAs.

2. The method of claim 1, wherein the RE DNAs present in c) are detected by PCR and/or sequencing of said RE DNAs.

3. The method of claim 2, wherein the RE DNAs present in c) are quantitated by said PCR and/or sequencing.

4. A method to selectively enrich for associated repetitive element (RE) DNAs in a sample comprising:
a) obtaining a blood or serum sample from a subject; and
b) enriching the sample of a) for extracellular vesicles (EVs),
c) isolating small nucleic acids from the enriched sample of b); and
d) quantitating said isolated small nucleic acids present in c).

5. A method to detect cancer in a subject comprising:
a) obtaining a blood or serum sample from the subject;
b) enriching the sample for EVs,
c) isolating small nucleic acids from b);
d) quantitating the RE DNAs present in c); and
e) comparing the level of RE DNAs in said sample to the level of RE DNAs in a control wherein the control does not have cancer,
wherein an increase in the levels of RE DNAs as compared to the control indicates the subject has cancer.

6. A method to monitor cancer treatment comprising:
a) obtaining a blood or serum sample from the subject;
b) enriching the sample for EVs,
c) isolating small nucleic acids from b);
d) quantitating RE DNAs present in c);
e) comparing the level of RE DNAs in said sample to the level of RE DNAs in a control wherein the control does not have cancer,
wherein an increase in levels of RE DNAs as compared to the control indicates the subject has cancer; and
f) repeating a) to e) at different times over the course of treatment or after treatment to determine the effect of treatment and/or the maintenance of remission in said subject.

7. The method of claim 1, wherein the sample is blood.

8. The method claim 1, wherein the sample is about 50 ul of blood.

9. The method of claim 1, wherein the repetitive DNA is at least one of HSATI, HSATII, L1P1 or Charlie 3.

10. The method of claim 3, wherein the RE DNA is quantitated as a proportion of RE DNA sequences to total nucleic acid sequences (including RE DNA, RE RNA non-RE gDNA and non-RE RNA).

11. The method of claim 3, wherein the RE DNA is quantitated as a ratio of one or more RE DNA sequences to one or more down-regulated non-RE sequences that co-purify with EVs.

12. The method of claim 5, wherein said subject with increased levels of RE DNA is treated for cancer.

13. The method of claim 12, wherein the cancer is Central Nervous System Cancers, Adult Ocular and Orbital (Ocular Adnexa) Tumors, Head and Neck Cancer, Thyroid Cancer, Endocrine and Neuroendocrine Tumors, Breast Cancer, Lung Cancer, Esophageal Cancer, Hepatocellular Carcinoma, Pancreatic Cancer, Biliary Tract Cancer, Gastric Cancer, Bladder Cancer, Prostate Cancer, Colorectal Cancer, Anal Cancer, Germ-Cell Cancer of the Testis and Related Neoplasms, Renal Cell Cancer, Ovarian, Fallopian Tube, and Primary Peritoneal Cancer, Uterine Cancer, Cervical Cancer, Carcinoma of the Vagina and Vulva, Gestational Trophoblastic Neoplasia, Non-Melanoma Skin Cancer, Malignant Melanoma, Primary Bone Tumors, Soft Tissue Sarcomas, Leukemias, Hodgkin Lymphoma, Non-Hodgkin Lymphoma, Multiple Myeloma, and/or pediatric cancers, including solid tumors such as bone and soft tissue sarcomas, neuronal cancers such as neuroblastoma, brain cancers, B and T-cell leukemias, myeloid leukemias, kidney, liver or eye cancers.

14. The method of claim 12, wherein the cancer is osteosarcoma.

15. The method of claim 12, wherein the cancer is breast cancer.

16. The method of claim 5, wherein the sample is blood.

17. The method of claim 5, wherein the sample is about 50 ul of blood.

18. The method of claim 5, wherein the repetitive DNA is at least one of HSATI, HSATII, L1P1 or Charlie 3.

19. The method of claim 5, wherein the RE DNA is quantitated as a proportion of RE DNA sequences to total nucleic acid sequences (including RE DNA, RE RNA non-RE gDNA and non-RE RNA).

20. The method of claim 5, wherein the RE DNA is quantitated as a ratio of one or more RE DNA sequences to one or more down-regulated non-RE sequences that co-purify with EVs.
